(19) United States
(12) Patent Application Publication
(10) Pub. No.: US 2006/0206960 A1
(43) Pub. Date: Sep. 14, 2006

(54) Higher plant cytosolic er-based glycerol-3-phosphate acyltransferase genes

(76) Inventors: Jitao Zou, Saskatoon (CA); Zhifu Zheng, Saskatoon (CA)

Correspondence Address: KIRBY EADES GALE BAKER, BOX 3432, STATION D, OTTAWA, ON, K1P 6N9, CA

(21) Appl. No.: 10/489,602

(22) PCT Filed: Sep. 23, 2002
(86) PCT No.: PCT/CA02/01445

§ 371 Date: Sep. 30, 2004

Related U.S. Application Data
(60) Provisional application No. 60/323,711, filed on Sep. 21, 2001.

(51) Int. Cl. A01H 1/00; C07H 21/04; C12N 9/10; C12N 15/82; C12N 5/04

(52) U.S. Cl. 800/281; 536/23.2; 435/193; 435/419; 435/468

(57) ABSTRACT

Glycerol-3-phosphate acyltransferase is the initial enzyme of the glycerolipid biosynthetic pathway. Biochemical analyses indicated that the reaction mediated by glycerol-3-phosphate acyltransferase represents a potential rate-limiting step for the synthesis of phospholipids and storage neutralipid, triacylglycerol. The present invention relates to the cloning of genes encoding extraplastidic membrane-bound glycerol-3-phosphate acyltransferases. Heterologous expression of the genes, GPAT1, GPAT2, and GPAT3 in a yeast glycerol-3-phosphate acyltransferase mutant demonstrated that the encoded products could efficiently utilize glycerol-3-phosphate to mediate sn-1 stereo-specific fatty acid acylation. The invention encompasses the glycerol-3-phosphate acyltransferase peptides disclosed and fragments and homologues thereof, the corresponding gene sequences and fragments and homologues thereof, as well as the use of the peptide and gene sequences of the present invention for use in generating recombinant proteins, and transgenic plants with altered lipid metabolism. In this way, the present invention also encompasses the use of such recombinant peptides and transgenic plants for the production of lipid products for use, for example, in pharmaceutical and nutritional applications.

### FIELD OF THE INVENTION

The present invention relates to genes and peptides involved in plant lipid biosynthesis. More particularly, the present invention relates to glycerol-3-phosphate (G-3-P) acyltransferase genes and the enzymes encoded thereby.

### BACKGROUND TO THE INVENTION

There is considerable commercial interest in the possibility of developing transgenic plants with altered lipid metabolism, which generate altered or increased yields of lipid products. The development of such modified plants and crops has significant potential for the development of nutritional and medicinal products. Therefore, the possibility of successfully engineering lipid-modified plants has implications upon both the agricultural and pharmaceutical industries. However, the metabolic pathways that regulate lipid metabolism in plants are not fully understood. Delineation of plant lipid metabolic pathways, and the generation of modified transgenic plants with beneficial characteristics, represents a considerable challenge to those of skill in the art.

Most fatty acids synthesized in an organism are incorporated into glycerolipids, either in the form of phospholipids that constitute the primary structural elements of membranes, or as triglycerides (TAG) stored in storage tissues as an energy source. In the course of glycerolipid biosynthesis, fatty acids are esterified into the sn-1 and sn-2 positions of the glycerol backbone by glycerol-3-phosphate acyltransferase (GPAT) and lyso-phosphatidic acid transferase (LPAT), respectively, to generate phosphatidic acid (PA). For the synthesis of storage lipids, diacylglycerol acyltransferase (DGAT) further acylates the sn-3 position of diacylglycerol, which is generated from PA through dephosphorylation, to produce TAG. In Arabidopsis thaliana, the synthesis of glycerolipids can occur both inside the plastids and the cytosolic membrane systems. The function of the plastidic glycerolipid pathway, also known as the “prokaryotic” pathway, is primarily confined to the synthesis of plastidic membrane lipids. The extraplastidic “eukaryotic” pathway generates phosphlipid species such as phosphatidylcholine (PC), phosphatidylethanolamine (PE), and phosphatidylinositol (Pl) for extraplastidic membranes. In addition, a portion of the diacylglycerol moiety of PC can also be channeled to the chloroplast envelope for the synthesis of thylakoid lipids. The synthesis of storage lipids (TAG) is believed to rely solely on the eukaryotic pathway.

Most naturally occurring glycerolipids, regardless from animals or plants, have an uneven distribution of fatty acids with regard to fatty acid chain length and desaturation. This non-random distribution of fatty acids in glycerolipids is governed by the composition of the fatty acyl-CoA pool as well as by the substrate specificities of the fatty acyltransferases. In both safflower and oil palm, microsomal-membrane GPAT exhibits preferences towards palmitoyl-CoA, while substrate specificity of the avocado GPAT was not apparent. The sn-2 acyltransferase LPAT of the eukaryotic pathway is known to be specific for unsaturated fatty acids and unable to utilize very long chain fatty acids such as eicosinoic and erusic-CoAs. Previous in vitro studies using preparations from safflower seeds suggested that DGAT has a broad substrate specificity of different acyl-CoAs and diacylglycerols. However, the Arabidopsis mutant AS11, which is defective in DGAT, has a phenotype of not only a reduced oil-content but also an altered fatty acid composition. Therefore, it becomes apparent that all three fatty acyltransferases contribute to the regulation of the overall fatty acid profiles of glycerolipids.

With perhaps the only exception of the chloroplast glycerol-3-phosphate acyltransferase, which is known to be a soluble protein, most fatty acyltransferases are integral membrane proteins that prove to be difficult to work with. Nonetheless, recent advances have generated a great deal of information about the molecular structures of LPAT and DGAT from a number of species including higher plants. Our knowledge on the cytosolic membrane-bound glycerol-3-phosphate acyltransferase, on the other hand, remains very limited. Although purification of ER-bound GPATs have been reported from a number of species, including cocoa, avocado, and oil palm, cloning of the ER GPAT gene has not been reported from any plant species. In view of the importance of GPAT catalyzing the initial and potentially one of the rate-limiting steps of the glycerolipid pathway, there is interest in cloning and manipulating GPAT activities in higher plants with a goal to alter the fatty acid compositions and to increase oil-content in seeds.

The enzymes considered responsible for the first committed step of glycerolipid biosynthesis in plants are glycerol-3-phosphate acyltransferases. Glycerol-3-phosphate acyltransferases catalyse the direct acylation of glycerol-3-phosphate with acyl Co—As to generate lysophosphatidic acid, which is an obligatory substrate for membrane as well as seed storage lipid biosynthesis. Therefore, the reaction mediated by glycerol-3-phosphate acyltransferases represents a potential rate-limiting step in the storage lipid biosynthesis, and thereby regulates seed oil-content. Higher plant glycerol-3-phosphate acyltransferases are known to have distinctive fatty acyl substrate specificities that are thought to be a key factor in determining stereo-specific distribution of fatty acid moieties in glycerolipids. Attempts in the purification of extraplastidic glycerol-3-phosphate acyltransferases have been fruitless for many years since the enzymes are tightly bound to cytosolic membrane systems and a reconstitution of active enzyme in vitro has not been achieved. So far, no plant extraplastidic glycerol-3-phosphate acyltransferase genes have been cloned.

Glycerolipid is a primary structural component of all cellular membrane systems and a highly valued storage product in the seed of several major crops. A significant body of literature shows that de novo glycerolipid biosynthesis involves a fundamental metabolic pathway that is well conserved in both prokaryotic and eukaryotic organisms (for review see Frentzen, 1993; Wilkison and Bell, 1997; Athenstaedt and Daum, 1999; Dircks and Sul, 1999). In plant cells, there are two prominent glycerolipid biosynthetic pathways compartmentalized in the plastid and the cytosolic membrane systems, termed the prokaryotic pathway and the eukaryotic pathways, respectively (Roughan and Slack, 1982; Heinz and Roughan, 1983; Browse et al., 1986). The plastidic glycerolipid pathway mainly contributes to the synthesis of plastidic membrane lipids (Roughan and Slack, 1984; Kunst et al., 1988). The extraplastidic “eukaryotic” pathway, on the other hand, plays a much broader role. Not only does it generate phospholipid species such as phosphatidylcholine (PC), phosphatidylethanolamine (PE), and phosphatidylinositol (PI) for extraplastidic membrane systems (Roughan and Slack, 1982; Moore, 1982), but also storage lipid in seeds (Harwood and Page, 1994). In addition, a portion of the diacylglycerol moiety of PC generated from the eukaryotic pathway is channeled to the chloroplast envelope for the synthesis of thylakoid lipids (Dörmann and Benning, 2002). An Arabidopsis mutant act 1, which lacks the plastidic pathway due to a deficiency in the chloroplast GPAT, does not exhibit any abnormal phenotype in growth or development (Kunst et al., 1988). Therefore, the eukaryotic pathway is able to compensate for the loss of the prokaryotic pathway, and it can be argued that the eukaryotic pathway plays a more dominant role in general plant lipid metabolism.

In both the prokaryotic and the eukaryotic glycerolipid pathways, the first committed step is, as previously mentioned, the fatty acid acylation at the sn-1 position of glycerol-3-phosphate (G-3-P) mediated by G-3-P acyl transferase (GPAT) (Bertrams and Heinz, 1976). The plastidic GPAT is a soluble protein that has been extensively studied (Bertrams and Heinz, 1981; Ishizaki et al., 1988; Murata et al., 1992). In contrast, the microsomal GPAT is a membrane-bound protein which has proven difficult to obtain in high purity and to retain activity in vitro. Only partial purification of the protein has been reported in a number of species, including cocoa (Fritz et al., 1986), avocado (Eccleston and Harwood, 1995), and oil palm (Manaf and Harwood, 2000). The microsomal GPAT is generally considered to be one of the rate-limiting enzymes involved in glycerolipid biosynthesis (Ichihara et al., 1987; Sun et al., 1988). Moreover, considerable attention has been focused on the properties of GPAT in relation to its role in determining the fatty acid composition of seed oil (Sun et al., 1988; Barfor et al., 1990; Hares and Frentzen, 1991). In most plants, the membrane-bound GPAT was found to exhibit preference towards saturated fatty acid (Ichihara, 1984; Griffiths et al., 1985; Frentzen, 1990; Manaf and Harwood, 2000). These studies have significantly advanced our understanding of the enzyme properties of the membrane-bound GPATs, but molecular characterization of the corresponding genes has proven elusive.

Genetic approach and mutant characterization continue to be instrumental in acquiring knowledge on plant lipid biochemistry. So far, the only mutant known to be defective in the eukaryotic glycerolipid pathway is that of the diacyglycerol acyltransferase (DGAT) mutant, TAG1 (Routaboul et al., 1999; Zou et al., 1999). In addition to having a low oil-content and modified fatty acid profile in seed (Katavic et al., 1995), the TAG1 mutant is also delayed in germination (Routaboul et al., 1999). There are no reports of mutants impaired in membrane GPAT. A GPAT deficiency in the eukaryotic pathway may implicate a broader range of biological processes since GPAT reaction is essential for membrane as well as storage lipid synthesis.

There is a continuing need develop systems and techniques for the efficient generation of lipid products for use in many industries, including the agricultural and pharmaceutical industries. In particular, there is a continuing need to isolate and characterize novel plant genes that are suitable for use in the production of lipids that are normally derived from plants. Such genes include those suitable for use in ex vivo lipid productions or genes that are suitable for use in engineering transgenic plants exhibiting modified lipid metabolism. Such plants may include crops having increased nutritional value, or crops that may be harvested for subsequent purification and use of the lipids, for example, as nutritional or pharmaceutical agents.

### SUMMARY OF THE INVENTION

It is an object of the present invention to isolate and characterize genes involved in plant lipid metabolism.

It is a further object of the present invention to provide a means for modifying lipid metabolism in plants, preferably by increasing or otherwise altering the yield of useful lipid-based products in the plants.

It is a further object of the present invention to provide a transgenic plant with increased levels of triacyl glycerol (TAG), which can be harvested for use in the production of, for example, nutritional and pharmaceutical products.

It is another object of the present invention to provide a means of increasing the levels of TAG in designated regions or organs of a plant, for specific commercial purposes. These commercial purposes may include, but are not limited to, the production of crops with increased pesticide resistance, crops with altered cross-breeding activity, plants with increased levels of lipid products concentrated in regions that permit facile harvesting and extraction.

It is a further object of the present invention to provide isolated recombinant proteins involved in plant lipid metabolism, which can be used in the commercial ex vivo production of TAG.

The inventors of the present application have succeeded in isolating and purifying gene sequences of glycerol-3-phosphate acyltransferase genes. The sequences of these genes have permitted the characterization of the corresponding protein products, which function as glycerol-3-phosphate acyltransferases. Corresponding transgenic plants have strong potential for the generation of crops having altered lipid metabolism for use in various industrial applications.

In accordance with one aspect of the invention there is provided an isolated nucleotide sequence, characterized in that said sequence encodes a cytoslic ER-based glycerol-3-phosphate acyltransferase protein, or a fragment thereof.

In accordance with another aspect of the invention there is provided an isolated nucleotide sequence characterized in that said isolated nucleotide sequence is selected from:

a) a cytoslic ER-based glycerol-3-phosphate acyltransferase gene as shown in SEQ ID NO: 1, 2 or 3, or a complement thereof;

b) a nucleotide sequence encoding a peptide with at least 40%, preferably at least 70%, more preferably at least 90%, more prefereably at least 95%, most preferably at least 99% identity to a peptide encoded by the nucleotide sequence of b);

wherein said nucleotide sequence or complement thereof encodes a protein or a part thereof, that alters a lipid metabolism of a transgenic plant exogenously expressing said nucleotide sequence compared to an unmodified plant.

In accordance with another aspect of the invention there is provided an isolated nucleotide sequence characterized in that said isolated nucleotide sequence is selected from the group consisting of:

a) a cytoslic ER-based glycerol-3-phosphate acyltransferase gene according to SEQ ID NO: 1, 2 or 3, or a complement thereof;

b) a nucleotide sequence that hybridizes under stringent conditions to the nucleotide sequence of a);

wherein said nucleotide sequence or complement thereof encodes a protein or part thereof that alters lipid metabolism of a transgenic plant exogenously expressing said nucleotide sequence compared to an unmodified plant.

Preferably, expression of said nucleotide sequence of the present invention confers on said transgenic plant an increased level of triayl glycerol (TAG) compared to an unmodified plant. More preferably, expression of said nucleotide sequence in a plant causes said plant to have seeds with an increased TAG level compared to an unmodified plant.

In accordance with another aspect of the invention there is provided an isolated and purified peptide characterized in that said isolated and purified peptide is encoded by the nucleotide sequence of the present invention.

In further aspects, the invention further pertains to a transgenic plant characterized in that said transgenic plant is derived from regeneration of a plant cell comprising an expression construct comprising a nucleotide sequence as described herein.

In accordance with another aspect of the invention there is provided a method of genetically modifying a plant, characterized in that the method comprising the steps of:

(a) introducing into a plant cell capable of being transformed and regenerated into a whole plant a construct comprising, in addition to the DNA sequences required for transformation and selection in plants, a nucleotide sequence according to present invention, operably linked to a promoter; and

(b) recovery of a plant which contains said nucleotide sequence.

In accordance with one aspect of the invention there is provided a method of identifying and isolating a DNA sequence substantially homologous to the nucleotide sequence of the present invention, characterized in that said method comprising the steps of:

synthesizing a degenerate oligonucleotide primer than can hybridize to the nucleotide sequence of the present invention under stringent conditions;

labelling said degenerate oligonucleotide primer; and

using said labelled degenerate oligonucleotide primer as a probe to screen a DNA library for said substantially homologous DNA sequence.

In accordance with another aspect of the invention there is provided a pair of primers characterized in that said primers hybridize to selected portions of the nucleotide sequence of the present invention, for amplifying a region of DNA between said primers by polymerase chain reaction.

In accordance with another aspect of the invention there is provided a method of producing a transgenic plant with altered lipid metabolism and/or altered lipid levels compared to an unmodified plant, characterized in that the method comprising the steps of:

(a) introducing into a plant cell capable of being transformed and regenerated into a whole plant a construct comprising, in addition to the DNA sequences required for transformation and selection in plants, a nucleotide sequence derived from a cytoslic ER-based glycerol-3-phosphate acyltransferase gene operably linked to a promoter; and

(b) recovery of a plant which contains said nucleotide sequence and has a modified lipid metabolism and/or growth potential compared to an unmodified plant.

In preferred aspects of the invention, the aforementioned methods may involve the use of various types of promoters, and the cytoslic ER-based glycerol-3-phosphate acyltransferase gene may be oriented for sense or anti-sense expression from the promoter.

In accordance with another aspect of the invention there is provided a method of identifying a plant that has been successfully transformed with a construct, characterized in that the method comprises the steps of:

(a) introducing into plant cells capable of being transformed and regenerated into whole plants a construct comprising, in addition to the DNA sequences required for transformation and selection in plants, a nucleotide sequence derived from a cytoslic ER-based glycerol-3-phosphate acyltransferase gene and encoding at least part of a cytoslic ER-based glycerol-3-phosphate acyltransferase gene product, operably linked to a promoter;

(b) regenerating said plant cells into whole plants; and

(c) inspecting the plants to determine those plants successfully transformed with said construct, and expressing said nucleotide sequence, said plants having an altered lipid content and/or an altered growth potential compared to an unmodified plant.

In another aspect, the present invention pertains to a bicistronic vector characterized in that said bicistronic vector comprises a first nucleotide sequence of the invention operatively linked to a first tissue-specific promoter, and a second nucleotide sequence of the invention operatively linked to a second tissue-specific promoter. Preferably, expression of said vector in a transgenic plant induces alternative lipid metabolism and growth potential characteristics in different tissues of said plant according to said first and second nucleotide sequences and said operatively linked first and second promoters. In one aspect said first nucleotide sequence is oriented in a sense direction relative to said first promoter, and said second nucleotide sequence is oriented in an antisense direction relative to said second promoter.

In another aspect said first nucleotide sequence encodes a biologically active form of a cytoslic ER-based glycerol-3-phosphate acyltransferase protein or a part thereof, and said second nucleotide sequence encodes a biologically inactive form of a cytoslic ER-based glycerol-3-phosphate acyltransferase protein or a part thereof. The invention also encompasses a transgenic plant characterized in that said transgenic plant is transformed with the aforementioned bicistronic vector.

The present invention also provides for a transgenic plant transformed with a construct comprising a DNA molecule encoding a glycerol-3-phosphate acyltransferase or functional part thereof. Preferably, the transgenic plants of the present invention have altered lipid metabolism, and preferably comprise a higher concentration of TAG. In an alternative embodiment the transgenic plants may be transformed with a construct encoding the glycerol-3-phosphate acyltransferase enzymes encompassed by the present invention, under the control of an organ-specific promoter. In this way, the glycerol-3-phosphate acyltransferase s are expressed in a plant organ of choice. In a further preferred embodiment, the plant enzymes and transgenic plant systems of the present invention may be suitable for the production of TAG for nutritional or pharmaceutical purposes. In addition, the transgenic plants of the present invention may exhibit alternative desirable properties such as altered developmental and growth characteristics resulting from their altered lipid metabolism. In another preferred embodiment, the present invention provides for a construct suitable for transforming a plant, wherein the construct can generate the expression of antisense mRNA for the polynucleotide molecules of the present invention, thereby reducing the level of glycerol-3-phosphate acyltransferase expression in the plant, as required.

In accordance with the present invention, there is therefore provided a means of generating TAG for use as nutritional and pharmaceutical agents. The TAG may be purified from the transgenic plants of the present invention using methods that are well known in the art, or may be generated ex vivo using recombinant proteins as defined by the present invention. In alternative embodiments, the transgenic plants of the present invention may bear fruit or seeds with increased levels of TAG.

In another aspect of the invention, there is provided a cytoslic ER-based glycerol-3-phosphate acyltransferase protein, or a fragment thereof that exhibits cytoslic ER-based glycerol-3-phosphate acyltransferase activity, characterized in that said protein comprises all of the peptide sequence motifs of: SEQ ID NO: 7, 8, 9, 10, 11, 12, 13, or 14. The invention also provides for nucleotide sequences expressing such peptides, and correspondingly transformed plant cells, plant materials (including seeds) and whole plants.

### BRIEF DESCRIPTION OF THE DRAWINGS

FIG. 1 provides a. nucleotide and b. amino acid sequences of GPAT1.

FIG. 2 provides a. nucleotide and b. amino acid sequences of GPAT2.

FIG. 3 provides Northern analysis of GPAT1 and GPAT2 gene expression. R=root, S=seedling, L=leaf, FB=flower bud, Q=silique

FIG. 4 illustrates fatty acyl specificity of GPAT1 and GPAT2. y-axis=GPAT activity, x-axis=fatty acyl donor. C16:0, palmitoyl-CoA; C16:1, palmitoleoyl-CoA; C18:0, stearyol-CoA; C18:1, oleoyl-CoA; C20:1, eicosenoic acyl-CoA.

FIG. 5A Genome structure of the gpat1-1 locus. LB, T-DNA left border. RB, T-DNA right border. Gray horizontal boxes represent exons. The T-DNA insert is not drawn to scale.

FIG. 5B Southern blot analysis of gpat1-1 genomic DNA digested with EcoR I and Spe I, and probed with the BAR gene.

FIG. 5C Northern analysis of GPAT1 transcript in Wt and gpat1-1. Approximately 10 μg of total RNA from siliques was loaded in each lane.

FIG. 6 Seed yield and storage lipid biosynthesis in Wt and gpat1-1 plants. (A) Average seed number in a mature silique. y-axis=average seeds per silique (B) Average weight of Wt and gpat1-1 seed. y-axis=average weight (mg) of 1000 seeds (C) Oil content of Wt and gpat1-1 seed. y-axis=oil content (percentage weight of seed) (D) Fatty acid composition of Wt and gpat1-1seed. x-axis=fatty acids, y-axis=Mol %

### DEFINITIONS

A “cytosolic ER-based GPAT” includes all proteins or fragments thereof that exhibit glycerol-3-phosphate acyltransferase activity and can reside in the endoplasmic reticulum of the cytosol when expressed in a eukaryotic cell. This encompasses GPAT proteins that are exclusively targeted to the ER, as well as GPAT proteins that are targetted both to the ER and additional cellular compartments.

The singular forms “a,” “an,” and “the” include plural reference unless the context clearly dictates otherwise.

A “coding sequence” or “coding region” is the part of a gene that codes for the amino acid sequence of a protein, or for a functional RNA such as a tRNA or rRNA. A coding sequence typically represents the final amino acid sequence of a protein or the final sequence of a structural nucleic acid. Coding sequences may be interrupted in the gene by intervening sequences, typically intervening sequences are not found in the mature coding sequence.

A “polynucleotide encoding an amino acid sequence” refers to a nucleic acid sequence that encodes the genetic code of at least a portion of a mature protein sequence, typically a contiguous string of amino acids typically linked through a peptide bond. An “amino acid sequence” is typically two or more amino acid residues, more typically 10 or more amino acids in a specific defined order.

A “complement” or “complementary sequence” is a sequence of nucleotides which forms a hydrogen-bonded duplex with another sequence of nucleotides according to Watson-Crick base-pairing rules. For example, the complementary base sequence for 5′-AGCT-3′ is 3′-TCGA-5′.

“Expression” refers to the transcription of a gene into structural RNA (rRNA, tRNA) or messenger RNA (mRNA) with subsequent translation into a protein in the case of the mRNA.

Polynucleotides are “functionally equivalent” if they perform substantially the same biological function. By substantially the same biological function it is meant that similar protein activities or protein function are encoded by a mRNA polynucleotide, or a structural polynucleotide has a similar structure and biological activity.

Polynucleotides are “heterologous” to one another if they do not naturally occur together in the same arrangement in the same organism. A polynucleotide is heterologous to an organism if it does not naturally occur in its particular form and arrangement in that organism.

Polynucleotides or polypeptides have “homologous” or “identical” sequences if the sequence of nucleotides or amino acid residues, respectively, in the two sequences is the same when aligned for maximum correspondence as described herein. Sequence comparisons between two or more polynucleotides or polypeptides are generally performed by comparing portions of the two sequences over a portion of the sequence to identify and compare local regions. The comparison portion is generally from about 20 to about 200 contiguous nucleotides or contiguous amino acid residues or more. The “percentage of sequence identity” or “percentage of sequence homology” for polynucleotides and polypeptides, such as 50, 60, 70, 80, 90, 95, 98, 99 or 100 percent sequence identity may be determined by comparing two optimally aligned sequences which may or may not include gaps for optimal alignment over a comparison region, wherein the portion of the polynucleotide or polypeptide sequence in the comparison may include additions or deletions (i.e., gaps) as compared to the reference sequence (which does not comprise additions or deletions) for optimal alignment of the two sequences.

The percentage of homology or similarity is calculated by: (a) determining the number of positions at which the identical nucleic acid base or amino acid residue occurs in both sequences to yield the number of matched positions; (b) dividing the number of matched positions by the total number of positions in the window of comparison; and, (c) multiplying the result by 100 to yield the percentage of sequence identity.

Optimal alignment of sequences for comparison may be conducted by computerized implementations of known algorithms, or by inspection. Readily available sequence comparison and multiple sequence alignment algorithms are, respectively, the Basic Local Alignment Search Tool (BLAST) (Altschul, S. F. et al 1990. J. Mol. Biol. 215:403; Altschul, S. F. et al 1997. Nucleic Acids Res. 25: 3389-3402) and ClustalW programs. BLAST is available on the Internet at http://www.ncbi.nlm.nih.gov and a version of ClustalW is available at http://www2.ebi.ac.uk. Other suitable programs include GAP, BESTFIT, FASTA, and TFASTA in the Wisconsin Genetics Software Package (Genetics Computer Group (GCG), 575 Science Dr., Madison, Wis.). For greater certainty, as used herein and in the claims, “percentage of sequence identity” or “percentage of sequence homology” of amino acid sequences is determined based on optimal sequence alignments determined in accordance with the default values of the BLASTX program, available as described above.

Sequence identity typically refers to sequences that have identical residues in order, whereas sequence similarity refers to sequences that have similar or functionally related residues in order. For example an identical polynucleotide sequence would have the same nucleotide bases in a specific nucleotide sequence as found in a different polynucleotide sequence. Sequence similarity would include sequences that are similar in character for example purines and pyrimidines arranged in a specific fashion. In the case of amino acid sequences, sequence identity means the same amino acid residues in a specific order, where as sequence similarity would allow for amino acids with similar chemical characteristics (for instance basic amino acids, or hydrophobic amino acids) to reside within a specific order.

The terms “stringent conditions” or “stringent hybridization conditions” includes reference to conditions under which a probe will hybridize to its target sequence, to a detectably greater degree than other sequences (e.g. at least 2-fold over background). Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH at which 50% of a complementary target sequence hybridizes to a perfectly matched probe. Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide. Exemplary low stringency conditions include hybridization with a buffer solution of 30% formamide, 1 M NaCl, 1% SDS at 37° C., and a wash in 2×SSC at 50° C. Exemplary high stringency conditions include hybridization in 50% formamide, 1 M NaCl, 1% SDS at 37° C., and a wash in 0.1×SSC at 60° C. Hybridization procedures are well-known in the art and are described in Ausubel et al., (Ausubel F. M., et al., 1994, Current Protocols in Molecular Biology, John Wiley & Sons Inc.).

“Isolated” refers to material that is: (1) substantially or essentially free from components which normally accompany or interact with it as found in its naturally occurring environment; or (2) if in its natural environment, the material has been non-naturally altered to a composition and/or placed at a locus in the cell not native to a material found in that environment. The isolated material optionally comprises material not found with the material in its natural environment. For example, a naturally occurring nucleic acid becomes an isolated nucleic acid if it is altered, or if it is transcribed from DNA which is altered, by non-natural, synthetic methods performed within the cell from which it originates.

Two DNA sequences are “operably linked” if the linkage allows the two sequences to carry out their normal functions relative to each other. For instance, a promoter region would be operably linked to a coding sequence if the promoter were capable of effecting transcription of that coding sequence and said coding sequence encoded a product intended to be expressed in response to the activity of the promoter.

A “polynucleotide” is a sequence of two or more deoxyribonucleotides (in DNA) or ribonucleotides (in RNA).

A “DNA construct” is a nucleic acid molecule that is isolated from a naturally occurring gene or which has been modified to contain segments of nucleic acid which are combined and juxtaposed in a manner which would not normally otherwise exist in nature.

A “polypeptide” is a sequence of two or more amino acids.

A “promoter” or transcriptional regulatory region is a cis-acting DNA sequence, generally located upstream of the initiation site of a gene, to which RNA polymerase may bind and initiate correct transcription.

A “recombinant” polynucleotide, for instance a recombinant DNA molecule, is a novel nucleic acid sequence formed through the ligation of two or more nonhomologous DNA molecules (for example a recombinant plasmid containing one or more inserts of foreign DNA cloned into it).

“Stress tolerance” refers to any type of stress that a plant may have to endure, and the capacity of such plant to tolerate the stress. The stress may be selected from a group including, but not limited to, heat, cold, frost, drought, flood, high winds etc. The stress may also be induced by other external factors including pest infestation and plant disease. Therefore the term “stress” further encompasses such insults. Stress tolerance relates to the capacity of a plant to cope with any such stresses without excessive damage and/or death.

“Growth potential” refers to the present and future ability of a plant to exhibit increased growth or vigour. Such growth may pertain to the entire biomass of the plant, but may also relate to the growth of specific organs. Increased growth or vigour relates to the rate at which a particular plant or plant organ changes weight. Typically such change in weight will be a gain in weight, but in certain in circumstances may also pertain to a loss in weight where desirable.

“Transformation” means the directed modification of the genome of a cell by the external application of recombinant DNA from another cell of different genotype, leading to its uptake and integration into the subject cell's genome.

A “transgenic plant” encompasses all descendants, hybrids, and crosses thereof, whether reproduced sexually or asexually, and which continue to harbour the foreign DNA.

### DETAILED DESCRIPTION OF THE PREFERRED EMBODIMENTS

In higher plants, de novo glycerolipid biosynthesis in the ER is required for the synthesis of glycerolipid components in membrane systems, and is also mainly, if not solely, responsible for the accumulation of storage lipid in seed. The present application identifies and characterizes plant genes encoding membrane-bound extraplastidic GPAT. The genes share no identifiable sequence similarity with the soluble chloroplast GPAT. The present application provides important evidence for a key role for GPAT as a factor controlling glycerolipid biosynthesis. As will be apparent from the present disclosure, the inventors have demonstrated that a reduction of glycerolipid content is evident in flower buds as well as in seeds of a gpat1-1 mutant. The corresponding studies of the metabolic defects of the gpat1-1 mutant provide, for the first time, conclusive evidence that membrane-bound GPAT plays an important role in plant lipid biosynthesis, and therefore identifies GPAT as a strategic target in genetic engineering for oilseed crop improvement.

The present application discloses the discovery of extraplastidic membrane bound glycerol-3-phosphate acyltransferase genes and their corresponding proteins, the first such enzymes to be identified from any plant species. The present invention also relates to homologous genes and their corresponding peptide sequences identifiable through the information embodied in the present application by persons skilled in the art for identification of membrane-bound extraplastidic glycerol-3-phosphate acyltransferase genes from other species. The present invention further encompasses the utility of such glycerol-3-phosphate acyltransferase genes as a molecular tool to alter the activity of glycerol-3-phosphate acyltransferases in plants, or to use such glycerol-3-phosphate acyltransferase genes as a molecular marker for breeding purposes. The present invention further encompasses the strategy of altering lipid biosynthesis through transgenic manipulation of glycerol-3-phosphate acyltransferase genes.

For the purposes of the present invention, the nucleic acid sequences encompassed by the application include those sequences encoding a peptide having at least 40% identity to a peptide encoded by SEQ ID NO: 1, 2, or 3, or a part thereof, providing that the nucleic acid sequence retains the capacity to alter lipid metabolism in a plant exogenously expressing the nucleic acid sequence.

Therefore, whilst the present invention discloses peptides sequences corresponding to closely related GPAT genes, it will be understood art that homologous sequences with significant predicted amino acid sequence identity to SEQ ID NOS: 1, 2, or 3, can be readily obtained in accordance with the teachings of the present invention. In this regard, nucleotide sequences of the present invention can be used to produce (degenerate) nucleotide probes, for the purposes of screening cDNA and genomic DNA libraries of various plant species. Related techniques are well understood in the art, for example as provided in Sambrook et al., Molecular Cloning: A Laboratory Manual, Cold Spring Harbor Press, Cold Spring Harbor, N.Y. (1989). In this way, sequences homologous to those of the present application are readily obtainable. For this reason, it is the intention of the present invention to encompass polynucleotide molecules comprising DNA sequences that encode peptides with significant sequence identity to those disclosed in the present application, wherein probes designed from SEQ ID NOS. 1, 2, or 3 or parts thereof, are utilized as polynucleotide probes to search for and isolate homologous polynucleotide molecules. Moreover, polynucleotides encoding proteins with significant sequence identity to those of the present application are expected give rise to similar protein products with similar biochemical characteristics, to those described in the present application.

Similar nucleic acid fragments may therefore be characterized by their ability to hybridize. Estimates of such homology are provided by either DNA-DNA or DNA-RNA hybridization under conditions of stringency as is well understood by those skilled in the art (Hames and Higgins, Eds. (1985) Nucleic Acid Hybridization, JRL Press, Oxford, U.K.). Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. Further conditions that may be suitable are included in the ‘definitions’ section herein.

Further, it is apparent to one skilled in the art that the sequences of SEQ ID NOS: 1, 2, or 3 can be used to isolate related genes from various other plant species by the use of plant DNA databases and comparative computational means. The similarity or identity of two polypeptide or polynucleotide sequences is determined by comparing sequences. In the art, this is typically accomplished by alignment of the amino acid or nucleotide sequences and observing the strings of residues that match. The identity or similarity of sequences can be calculated by known means including, but not limited to, those described in Computational Molecular Biology, Lesk A. M., ed., Oxford University Press, New York, 1988, Biocomputing: Informatics and Genome Projects, Smith, D. W., ed., Academic Press, New York, 1993., Computer Analysis of Sequence Data, Part I, Griffin, A. M. and Griffin, H. G., eds., Humana Press, New Jersey, 1994 and other protocols known to those skilled in the art. Moreover, programs to determine relatedness or identity are codified in publicly available programs. One of the most popular programs comprises a suite of BLAST programs, three designed for nucleic acid sequences (BLASTN, BLASTX and TBLASTX), and two designed for protein sequences (BLASTP and TBLASTN) (Coulson, Trends in Biotechnology, 12:76-80, 1994). The BLASTX program is publicly available from NCBI and other sources such as the BLAST Manual, Altschul, S., et al., NCBI NLM NIH Bethesda Md. 20984, also http://www.ncbi.nlm.nih.gov/BLAST/blast_help.html) provides online help and further literature references for BLAST and related protein analysis methods, and Altschul, S., et al., J. Mol. Biol 215:403-410, 1990.

The isolated polynucleotide can be sequenced and the DNA sequence used to further screen DNA sequence collections to identify related sequences from other species. The DNA sequence collections can comprise EST sequences, genomic sequences or complete cDNA sequences.

Similar nucleic acid sequences of the present invention may also be characterized by the percent identity of the amino acid sequences that they encode to the amino acid sequences disclosed herein, as determined by algorithms commonly employed by those skilled in this art. Preferred are those nucleic acid fragments whose nucleotide sequences encode amino acid sequences that are 40%, 50%, identical to the amino acid sequences reported herein. More preferred nucleic acid fragments encode amino acid sequences that are 70% or 90% identical to the amino acid sequences reported herein. Most preferred are nucleic acid fragments that encode amino acid sequences that are 95% or 99% identical to the amino acid sequences reported herein. For example, sequence alignments and percent identity calculations can be performed using the Megalign program of the LASARGENE bioinformatics computing suite (DNASTAR Inc., Madison, Wis., U.S.A.). In another example, multiple alignment of the sequences can performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5.

The present invention therefore encompasses DNA sequences obtained by techniques known in the art for isolating homologous DNA sequences, including the use of degenerate oligonucleotide probes, and/or computer database homology searches. The degree of amino acid sequence identity will vary for each identified sequence. It is the intention of the present invention to encompass polynucleotide sequences comprising at least 40% sequence identity with regard to the peptide sequences encoded by the corresponding polynucleotides. Without wishing to be bound by theory, it is generally expected in the art that enzymes with at least 40% identity can be expected to have enzymatic activities that are similar in scope. In this regard, the essential structural features of the enzyme are preserved to scaffold the conformation of the catalytic site of the enzyme. Therefore, the present invention encompasses polynucleotide molecules derived by screening genomic and cDNA libraries of species other than Arabidopsis, using degenerate DNA probes derived from the sequences encompassed by the present application.

The present invention also encompasses polynucleotide sequences that encode peptides comprising at least 70% amino acid sequence identity to peptides encoded by SEQ ID NOS: 1, 2, or 3. In this regard, homologous proteins with at least 70% predicted amino acid sequence identity are expected to encompass proteins with similar glycerol-3-phosphate acyltransferase activity as those defined by the present invention, but perhaps with altered substrate specificity. Such proteins may be derived from similar species of plant.

The present invention also encompasses polynucleotide sequences encoding peptides comprising at least 90% or 99% sequence identity to the peptides encoded by SEQ ID NOS: 1, 2 or 3. This class of related proteins is intended to include close gene family members with very similar or identical catalytic activity. In addition, peptides with 90% to 99% amino acid sequence identity may be derived from functional homologues of similar species of plant, or from directed mutations to the sequences encompassed by the present application.

It will also be understood to a person of skill in the art that site-directed mutagenesis techniques are readily applicable to the polynucleotide sequences of the present invention. Related techniques are well understood in the art, for example as provided in Sambrook et al., Molecular Cloning: A Laboratory Manual, Cold Spring Harbor Press, Cold Spring Harbor, N.Y. (1989). In this regard, the present invention teaches the isolation and characterization of the DNA sequences as provided as SEQ ID NOS: 1, 2 and 3. However, the present invention is not intended to be limited to these specific sequences. Numerous directed mutagenesis techniques would permit the non-informed technician to alter one or more residues in the nucleotide, thus changing the subsequently expressed polypeptide sequences. Moreover, commercial ‘kits’ are available from numerous companies that permit directed mutagenesis to be carried out (available for example from Promega and Biorad). These include the use of plasmids with altered antibiotic resistance, uracil incorporation and PCR techniques to generate the desired mutation. The mutations generated may include point mutations, deletions and truncations as required. The present invention is therefore intended to encompass corresponding mutants of the peptides shown in SEQ ID NOS. 1, 2 and 3 and their corresponding genes.

The mutated variants of the sequences disclosed in the present application are predicted to include enzymes with reduced or increased glycerol-3-phosphate acyltransferase activity, as well as altered substrate specificity. Such mutants may confer advantageous properties to subsequently transformed transgenic cell lines and plants. For example, a transgenic plant comprising a construct overexpressing an inactive mutant of the enzymes of the present invention can be expected to have a significantly altered profile of lipid constituents, including a reduction in TAG content. In contrast, mutant glycerol-3-phosphate acyltransferase enzymes with increased catalytic turnover are expected to give rise to transgenic plants with an high level of TAG. Mutant glycerol-3-phosphate acyltransferase enzymes with altered substrate specificity will likely be useful in altering the relative quantities of lipid metabolism products generated in a correspondingly transformed plant.

The polynucleotide sequences of the present invention must be ligated into suitable vectors before transfer of the genetic material into plants. For this purpose, standard ligation techniques that are well known in the art may be used. Such techniques are readily obtainable from any standard textbook relating to protocols in molecular biology.

The DNA sequences encompassed by the present application are considered suitable for transformation into any plant variety to alter the lipid metabolism of the plant by expression of the sequences using a desired promoter. Particularly preferred examples of plant varieties include those cultivated for the production of oils. Typical examples include, but are not limited to, canola, safflower, sunflower, olive and various vegetables and fruits.

Numerous methods for plant transformation have been developed, including biological and physical, plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pages 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

Further methods that have general utility include Agrobacterium based systems, using either binary and cointegrate plasmids of both A. tumifaciens and A. rhyzogenies. (e.g., U.S. Pat. No. 4,940,838, U.S. Pat. No. 5,464,763), the biolistic approach (e.g., U.S. Pat. No. 4,945,050, U.S. Pat. No. 5,015,580, U.S. Pat. No. 5,149,655), microinjection, (e.g., U.S. Pat. No. 4,743,548), direct DNA uptake by protoplasts, (e.g., U.S. Pat. No. 5,231,019, U.S. Pat. No. 5,453,367) or needle-like whiskers (e.g., U.S. Pat. No. 5,302,523). Any method for the introduction of foreign DNA and/or genetic transformation of a plant cell may be used within the context of the present invention.

The following are examples of plant transformation techniques, which may be used in accordance with the teachings of the present invention. Other transformation techniques may also be used, as required.

A. Agrobacterium-mediated Transformation: One method for introducing an expression vector into plants is based on the natural transformation system of Agrobacterium. See, for example, Horsch et al., Science 227: 1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant. Sci. 10: 1 (1991). Descriptions of Agrobacterium vector systems and methods for Agrobacterium-mediated gene transfer are provided by Gruber et al., supra, Miki et al., supra, and Moloney et al., Plant Cell Reports 8: 238 (1989). Bechtold et al., C. R. Acad. Sci. Paris Life Sciences, 316:1194-9 (1993).

B. Direct Gene Transfer: Several methods of plant transformation, collectively referred to as direct gene transfer, have been developed as an alternative to Agrobacterium-mediated transformation. A generally applicable method of plant transformation is microprojectile-mediated transformation wherein DNA is carried on the surface of microprojectiles measuring 1 to 4 .mu.m. The expression vector is introduced into plant tissues with a biolistic device that accelerates the microprojectiles to speeds of 300 to 600 m/s which is sufficient to penetrate plant cell walls and membranes. Sanford et al., Part. Sci. Technol. 5: 27 (1987), Sanford, J. C., Trends Biotech. 6: 299 (1988), Klein et al., Bio/Technology 6: 559-563 (1988), Sanford, J. C., Physiol Plant 79: 206 (1990), Klein et al., Biotechnology 10: 268 (1992). See also U.S. Pat. No. 5,015,580 (Christou, et al), issued May 14, 1991; U.S. Pat. No. 5,322,783 (Tomes, et al.), issued Jun. 21, 1994.

Another method for physical delivery of DNA to plants is sonication of target cells. Zhang et al., Bio/Technology 9: 996 (1991). Alternatively, liposome or spheroplast fusion have been used to introduce expression vectors into plants. Deshayes et al., EMBO J., 4: 2731 (1985), Christou et al., Proc Natl. Acad. Sci. U.S.A. 84: 3962 (1987). Direct uptake of DNA into protoplasts using CaCl₂ precipitation, polyvinyl alcohol or poly-L-ornithine have also been reported. Hain et al., Mol. Gen. Genet.199: 161 (1985) and Draper et al., Plant Cell Physiol.23: 451 (1982). Electroporation of protoplasts and whole cells and tissues have also been described. Donn et al., In Abstracts of VIIth International Congress on Plant Cell and Tissue Culture IAPTC, A2-38, p 53 (1990); D'Halluin et al., Plant Cell 4: 1495-1505 (1992) and Spencer et al., Plant Mol. Biol. 24: 51-61 (1994).

Following transformation of target tissues, expression of the above-described selectable marker genes allows for preferential selection of transformed cells, tissues and/or plants, using regeneration and selection methods now well known in the art.

The foregoing methods for transformation would typically be used for producing a transgenic variety. The transgenic variety could then be crossed, with another (non-transformed or transformed) variety, in order to produce a new transgenic variety.

Alternatively, a genetic trait which has been engineered into a particular line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties which do not contain that gene. As used herein, “crossing” can refer to a simple X by Y cross, or the process of backcrossing, depending on the context. Once a transgenic plant has been established, it is important to determine the lipid content of the plant, or various plant organs.

Once transgenic plants have been selected, the lipid content can be analyzed, and lipids and TAG extracted from the plant material. Several techniques are known in the art to analyze the chemical content of the plant material, and in particular the lipid and TAG content. These techniques include gas chromatography (GC), high performance liquid chromatography and MS-GC, as well as other techniques that are familiar to those of skill in the art. Moreover, the lipid products may be extracted from the plant by any one of several techniques that are well known to those of skill in the art.

It is predicted that the glycerol-3-phosphate acyltransferase genes disclosed herein are the first glycerol-3-phosphate acyltransferase genes to be reported in plants.

The present invention therefore encompasses nucleotide sequences which include the GPAT gene sequence, or fragments thereof, of homologues thereof. As previously mentioned, such nucleotide sequences include, but are not limited to, the gene sequence indicated in SEQ ID NOS: 1, 2, or 3, and fragments thereof. Preferably, the nucleotide sequences of the invention have the capacity to alter plant metabolism, such that exogenous expression of the nucleotide sequence in a plant induces the plant to exhibit an altered lipid content compared to an unmodified plant. The sequences of the present invention further include the nucleotide and peptide sequences derived from the sequences shown in SEQ ID NOS: 1, 2, or 3.

In another embodiment of the present invention, the GPAT gene sequences, and parts, complements, and homologues thereof are used to modify plant lipid content by the transformation of plant cells with a plant transformation vector comprising a coding, for example, a region of a nucleic acid of SEQ ID NOS: 1, 2, or 3, or a homologue thereof, under the control of a heterologous or native/homologous promoter.

In another embodiment of the present invention, one or more portions, of at least 10 amino acids of the protein encoded by the nucleic acid sequence shown in SEQ ID NOS: 1, 2, or 3, or a complement thereof or a homologue thereof, are expressed in a host plant, said expression causing the lipid content of the plant.

In another embodiment of the present invention, the nucleic acid sequence of SEQ ID NO: 1, 2, or 3, or parts thereof or homologues thereof, is used to modify plant lipid content by the transformation of plant cells with a plant transformation vector comprising a coding region of said polynucleotide under the control of the promoter normally associated with the GPAT gene sequences. In alternative embodiments, the GPAT gene or a derivative thereof may be inserted into a construct under the control of a constitutive promoter such that the gene is expressed from low to high levels in all plant tissues of the transgenic plant. In this way, the modification of plant lipid content will be conferred to the entire plant. In further alternative embodiments, the GPAT gene or parts or homologues thereof may be inserted into a construct for plant transformation under the control of a tissue specific promoter. In this way, the modification of plant lipid content will be conferred only to selected tissues and organs of the plant. Alternatively, the promoter may be stress responsive, only activating exogenous expression of GPAT if certain conditions are met. Such conditions may include, but are not limited to, infestation, disease, or environmental conditions such as heat, cold, frost, drought, flood etc. Many such promoters are well known to those skilled in the art, and their use in conjunction with GPAT is intended to fall within the scope of the invention.

In one embodiment of the present invention, nucleic acids encoding a protein at least 40% identity to the protein sequence encoded by SEQ ID NO: 1, 2 or 3 are isolated by routine techniques as described herein, and said nucleic acids are used to alter the lipid content of the plant species from which they were derived by introduction of said nucleic acid or portion thereof into said plant species and recovering a plant wherein the phenotype of the plant has changed as a result of the introduction of the nucleic acid sequence, or portion thereof into the plant species.

In another embodiment of the present invention, said nucleic acids that encode a protein at least 40% identity to the protein encoded by the nucleotide sequence of SEQ ID NO: 1, 2 or 3 are used to alter the lipid content of a plant by introduction of said nucleic acid into a plant species heterologous to the plant species from which said nucleic acid sequence was derived.

In yet another embodiment of the present invention, a nucleic acid sequence of the present invention is used as a visible marker for plant transformation, said marker producing plants with an altered lipid content relative to plants not transformed with the same. In this way, plants may be conferred, for example, with specific plant organs having different sizes compared to those of an unmodified plant. This new feature can be used to select for only those plant successfully transformed with the construct. Also within the scope of the invention are bicistronic vectors comprising both a GPAT derived sequence, and an additional sequence or sequences for conferring additional modifications to the plant. It is the intention of the invention to encompass all such related plant selection techniques that utilize the GPAT gene, or parts thereof, or homologues thereof. The advantages of using selection systems that do not include antibiotic/herbicide resistance marker genes for producing transgenic plants are well recognized. Since GPAT expression generates one or more phenotypes that are readily distinguishable from wild type plants, it is possible to develop transformation vectors based on the GPAT gene that are devoid of any antibiotic or herbicide selection markers to provide a novel and very efficient alternative to the currently available selection systems.

In yet another embodiment of the present invention, the expression of an exogenous GPAT gene sequence is modified by the presence of an exogenous GPAT coding sequence. The exogenous GPAT coding sequence can be an altered form of the endogenous GPAT coding region normally found in said plant species, or a GPAT functional homologue from a different plant species. Expression of the exogenous GPAT protein may be expected to alter the activity of the native GPAT protein, or the exogenously produced GPAT protein can encode an activity that provides a phenotypic distinction. In another embodiment of the invention there is provided a method of expressing a GPAT gene sequence or derivative thereof in a plant species comprising the steps of:
a) Introducing into a plant cell capable of being transformed a genetic construct comprising a first DNA expression cassette that comprises, in addition to the DNA sequences required for transformation and selection in said cells, a DNA sequence derived from a GPAT gene, for example, that encodes a peptide having at least 40% identity to the peptide encoded by GPAT, operably linked to a suitable transcriptional regulatory region and,
b) recovery of a plant which contains said recombinant DNA, said plant exhibiting altered lipid metabolism relative to an unmodified plant.

The suitable transcriptional regulatory region can be the regulatory region normally associated with the GPAT gene or GPAT coding sequence, or a heterologous transcriptional regulatory region.

In another embodiment of the invention the subject method includes a method for modifying the lipid content of a plant comprising:
(a) introducing into a plant cell capable of being transformed and regenerated to a whole plant a genetic construct comprising a first DNA expression cassette that comprises, in addition to the DNA sequences required for transformation and selection in plant cells, a DNA sequence that comprises a polynucleotide region encoding a GPAT gene or a part thereof, operably linked to a suitable transcriptional regulatory region and,

(b) recovery of a plant which contains said recombinant DNA.

In this way the chimeric gene is introduced into a plant cell and a plant cell recovered wherein said gene is integrated into the plant chromosome. The plant cell is induced to regenerate and a whole plant is recovered with altered lipid metabolism. The present invention therefore encompasses plant cells and transgenic plants transformed with constructs comprising GPAT or derivatives thereof, and methods for their generation.

The use of gene inhibition technologies such as antisense RNA or co-suppression or double stranded RNA interference is within the scope of the present invention. In these approaches, the isolated gene sequence is operably linked to a suitable regulatory element.

Accordingly, in one embodiment of the invention the subject method includes a method to modify the stress response or growth potential of a plant comprising the steps of:
a.) Introducing into a plant cell capable of being transformed a genetic construct comprising a first DNA expression cassette that comprises, in addition to the DNA sequences required for transformation and selection in said cells, a DNA sequence that encodes a GPAT coding sequence encoding a protein or part thereof having at least 40% sequence identity to the protein encoded by the sequence of SEQ ID NO: 1, 2, or 3 at least a portion of said DNA sequence in an antisense orientation relative to the normal presentation to the transcriptional regulatory region, operably linked to a suitable transcriptional regulatory region such that said recombinant DNA construct expresses an antisense RNA or portion thereof of an antisense RNA; and
b.) recovery of a plant which contains said recombinant DNA.

It will be apparent to the skilled artisan that the polynucleotide encoding the sequence can be in the antisense (for inhibition by antisense RNA) or sense (for inhibition by co-suppression) orientation, relative to the transcriptional regulatory region, or a combination of sense and antisense RNA to induce double stranded RNA interference (Chuang and Meyerowitz, PNAS 97: 4985-4990, 2000, Smith et al., Nature 407: 319-320, 2000).

The present invention also encompasses the use of antisense expression to reduce the levels of GPAT within the plant, for example for the purposes of reducing the lipid content of the plant. This concept may be extended to the use of organ-specific promoters and/or the use of bicistronic vectors for modifying overall plant lipid content and/or architecture. In one example, a stalk specific promoter may be used with GPAT in an antisense direction. Conversely, a seed specific promoter may be used with GPAT in a sense direction, thereby increasing seed lipid content. Preferably, these two gene cassettes may both be incorporated into a single bicistronic vector. Transgenic plants having such a vector may exhibit short stalks for improved wind damage resistance, and yet may yield modified seeds with increased lipid content thereby improving productivity. Many more examples of GPAT sense/antisense expression with various organ or expression combinations specific promoters may be designed, all of which are intended to fall within the scope of the present invention.

The following examples serve to illustrate the method and in no way limit the utility of the invention.

### EXAMPLES

### Example 1

### Molecular Cloning of GPAT Genes

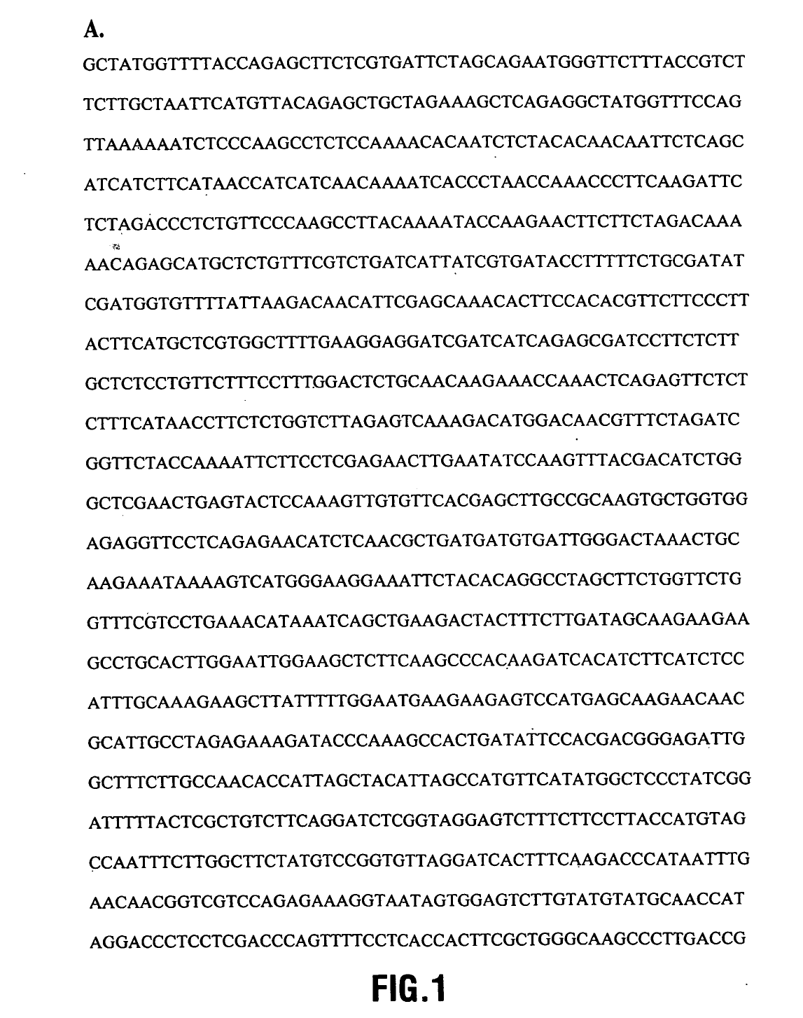

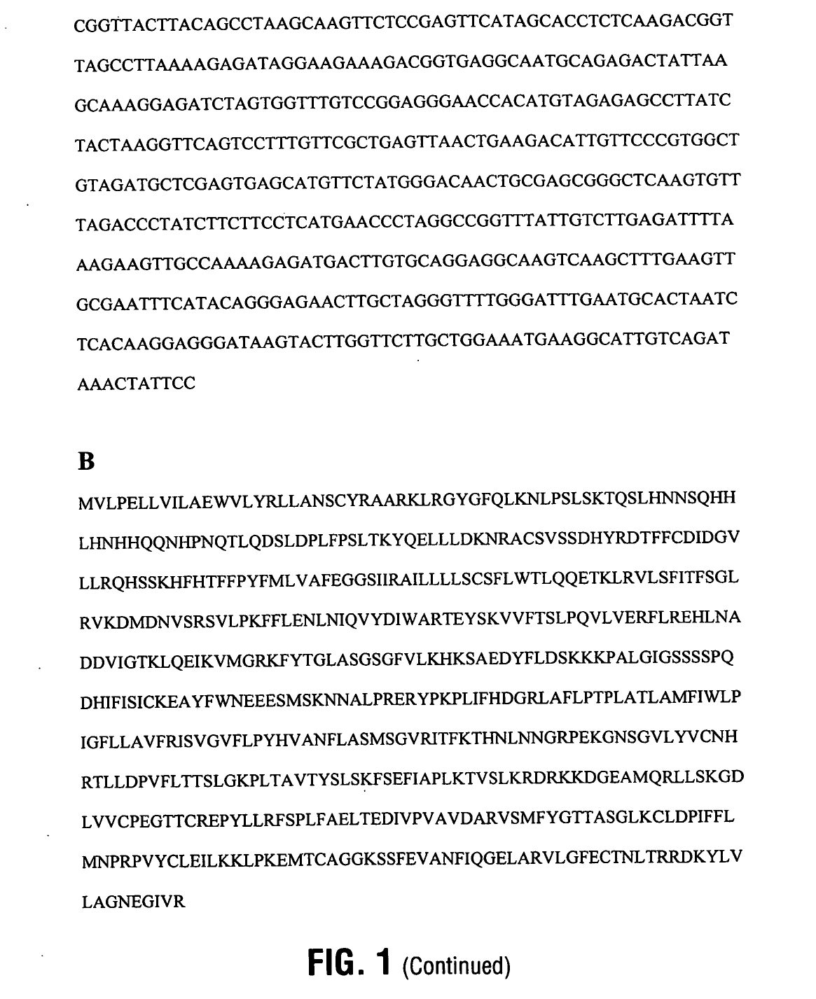

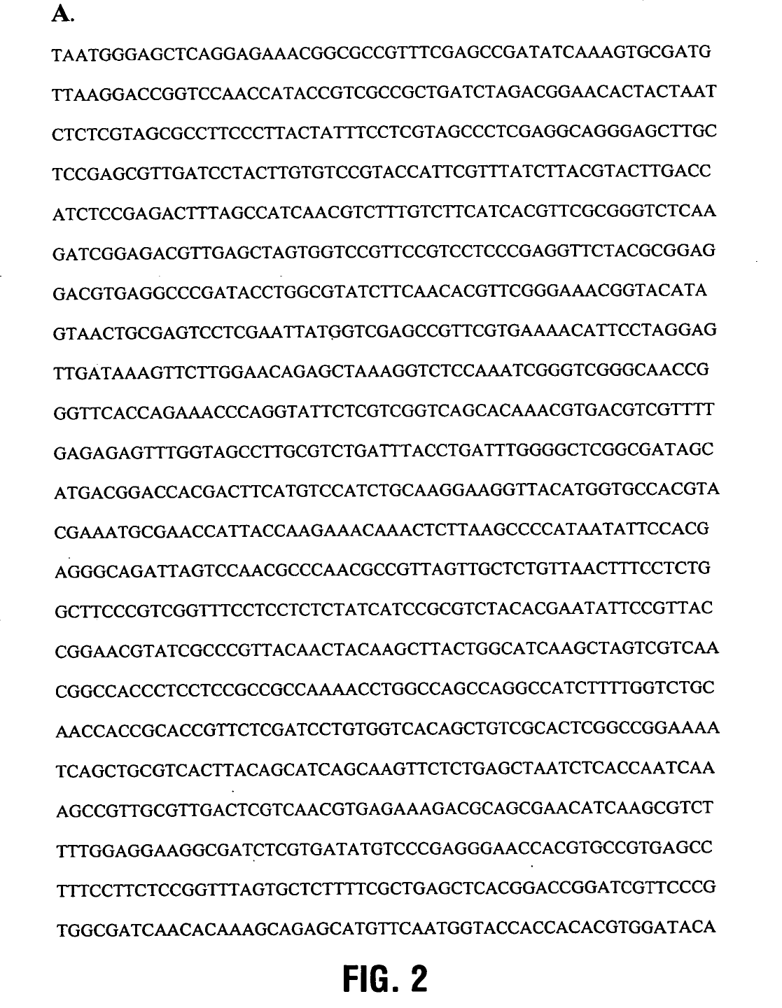

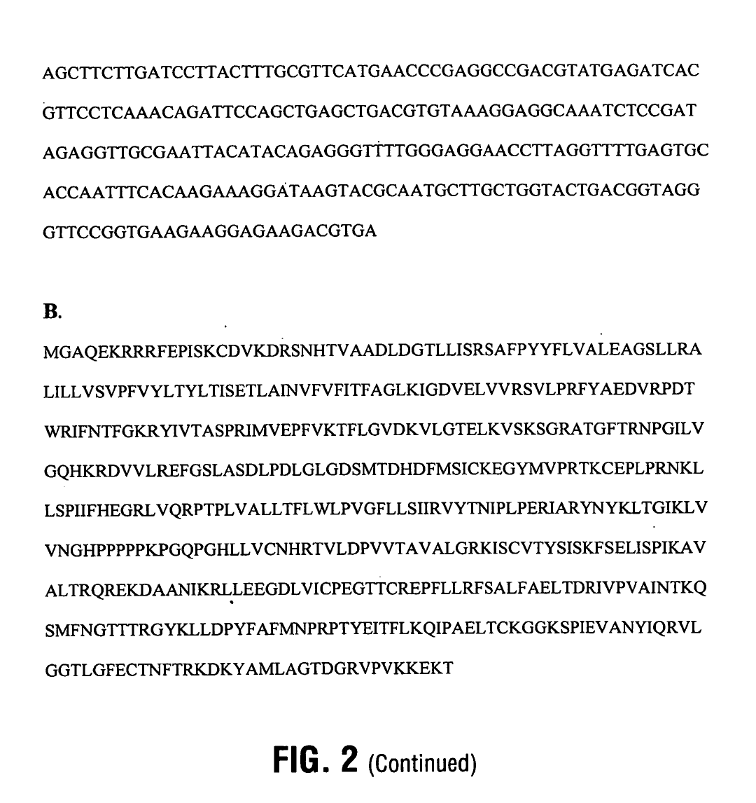

Based on our previous studies with the yeast ER-bound GPAT (Zheng and Zou, 2001) and the general structural information available from other membrane-bound sn-1 acyltransferases (Wilkison and Bell, 1997; Lewin et al., 1999), the inventors made the following assumptions. (i) A plant extraplastidic membrane GPAT will share certain conserved domains with other fatty acyl acyltransferases, and (ii) it will be significantly larger in size than the sn-2 or sn-3 acyltransferases. BLAST searching using the entire yeast GPAT sequences yielded no result in an attempt to identify a plant homologue. However, a series of BLAST searches (Altschul et al., 1990), utilizing a query from a partial sequence encompassing a region conserved between the yeast GPATs and other fatty acyltransferases (including the mammalian dihydroxyacetone phophate acyltransferase), identified a gene (F12K11.15, designated GPAT1) on chromosome 1 of Arabidopsis that encodes a putative protein of 585 aa. Using GPAT1 as a query, its homolog GPAT2 was also identified. The two cDNAs corresponding to GPAT1 and GPAT2 were cloned from Arabidopsis by RT-PCR and fully sequenced. The GPAT1 (FIG. 1) and GPAT2 (FIG. 2) are not structurally related to the plastidic GPAT (Nishida et al., 1993). Moreover, using software TopPred2, GPAT1 and GPAT2 were predicted to possess two transmembrane domains. Similar membrane-spanning predictions were also obtained using the TMpred program (Hofmann and Stoffel, 1993). Taken together, our results show that the Arabidopsis GPAT1 and GPAT2 represent a new type of membrane-bound G-3-P acyltransferase that are different from the previously identified soluble plastidic GPAT.

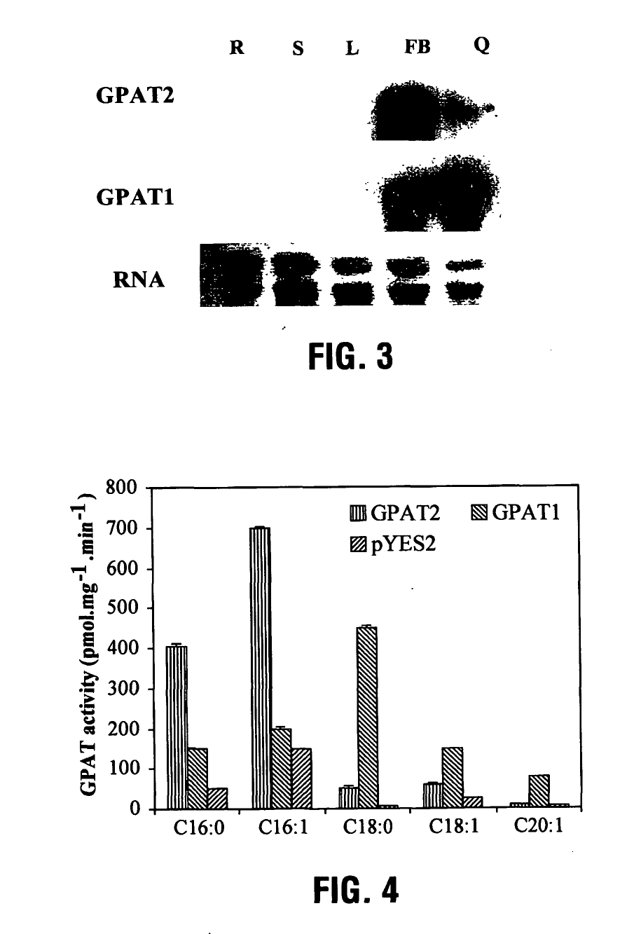

To determine the expression pattern of GPAT1 and GPAT2, northern analyses were performed with total RNA isolated from various Arabidopsis tissue. As shown in FIG. 3A, the highest level of steady-state transcript of GPAT1 was detected in siliques containing developing seeds. Abundant transcript was also present in flower buds. GPAT2 was found to be expressed mainly in flower buds, but some mRNA signal was also detectable in developing siliques (FIG. 3B). The tissue-specific expression pattern of the gene generally coincides with developmental stages where elevated glycerolipid synthesis is known to take place. Specifically, previous findings have demonstrated that the Arabidopsis flowers are rich in C₁₆ saturated fatty acids (C_{16:0}), while Arabidopsis seeds accumulate very long chain fatty acid (C_{20:1}).

GPAT1 is shown as SEQ ID NO: 1, and GPAT2 is shown as SEQ ID NO: 2 in the sequence listing. Moreover, the corresponding deduced GPAT1 peptide sequence is SEQ ID NO: 4, and the corresponding deduced GPAT2 peptide sequence is shown as SEQ ID NO: 5.

The inventors have also succeeded in isolated a third corresponding GPAT gene sequence designated accession number At1g01610. The GPAT3 gene sequence has been designated SEQ ID NO: 3, and the corresponding deduced protein sequence as SEQ ID NO: 6. The gene shows various characteristics (1) it shows 41% identity to GPAT1. (2) Heterologous expression in yeast showed that this gene encoded a protein which possessed GPAT activity; (3) In vitro translation and targeting assays revealed that the gene-encoded protein could be integrated into canine pancreatic microsomal membranes, strongly suggesting that this protein can be targeted in ER of plant cells. (4) Northern analysis show that the transcript accumulated in all tissues tested (root, seedling, leaf, flower, and silique), with the highest level in flower, silique and seedling.

### Example 2

### GPAT Genes Disclosed Encode G-3-P acyltransferases

In order to establish the biochemical function of plant GPAT, the inventors over-expressed the GPAT1 and GPAT2 cDNA in a yeast mutant gat1Δ via a multiple copy expression vector pYES-2. The gat1Δ strain harbors a mutation in its GAT1 gene that encodes a major ER-bound G-3-P acyltransferase in yeast. Due to the low residual GPAT activity, functionality of putative membrane-bound G-3-P acyltransferases can be readily tested in this strain (Zheng and Zou, 2001). The inventors assessed the G-3-P acylation activity of total yeast lysate prepared from the gat1Δ strain expressing GPAT1, GPAT2 and the control vector, respectively, using stearyol-CoA and ¹⁴C-labeled G-3-P as substrates. Separation of the lipid products of the reaction mixture by thin layer chromatography (TLC) showed an increased generation of lysophosphatidic acid (LPA). Phosphatidic acid (PA) formation was also increased in the reaction, a common observation in acyltransferase assays, due to the presence of an endogenous LPA acyltransferase (Manaf and Harwood, 2000). Since the incorporation of ¹⁴C-labeled G-3-P into all successive intermediates requires the initial enzyme activity of G-3-P acyltransferase, the increased radioactivity detected in the total lipid of the reaction mixture can only be attributed to the presence of the heterologously expressed plant GPAT genes. From these results the inventors can conclude that Arabidopsis GPAT1 and GPAT2 encode functional GPATs in Arabidopsis.

The biochemical properties of lipid biosynthesis related enzymes are crucially important in designing genetic engineering approaches to modify the fatty acid contents of lipids in plants. Most fatty acyltransferases characterized to date demonstrate a certain degree of fatty acid preference, and it is known that the fatty acyl substrate-specificities of acyltransferases are major determinants of glycerolipid fatty acid composition in plant tissues. The inventors therefore examined the substrate specificity of GPAT1 and GPAT2 using various fatty acyl donors. The calculation of enzyme specificity with respect to a particular fatty acyl CoA was based on the difference in G-3-P acyltransferase activity between the gat1Δ strain expressing the GPAT genes and the strain harboring the control vector. As shown in FIG. 4A, GPAT1 could utilize each of the fatty acyl-CoAs examined, including the very long chain eicosenoic acid. However, by far, the highest specific activity employing our assaying conditions was detected with stearyol-CoA. This result suggests that GPAT1 prefers stearyol-CoA to other fatty acyl donors. GPAT2, on the other hand, displayed an apparent preferences toward 16-carbon fatty acids, and unable to incorporate the very long chain fatty acids (FIG. 4B).

### Example 3

### Identification of a T-DNA Insertion Mutant gpat1-1

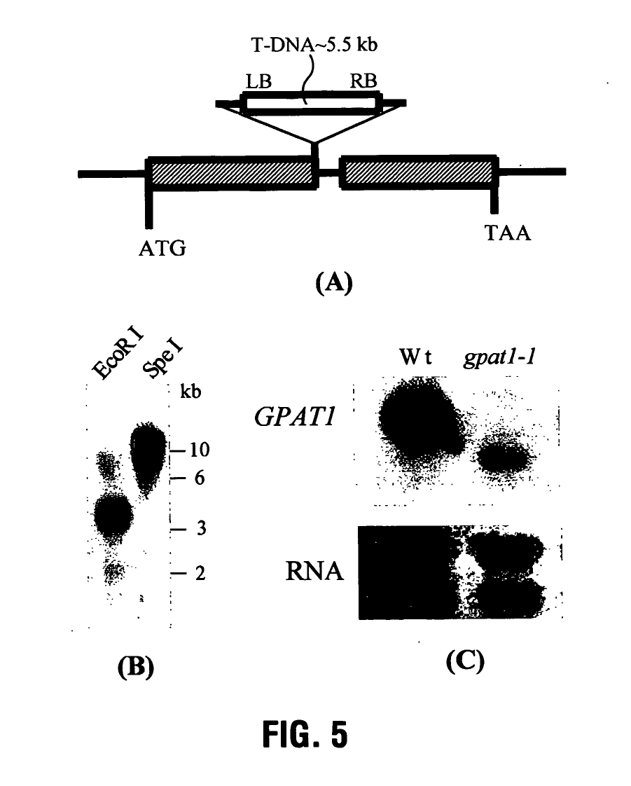

To investigate the functional significance of GPAT, the inventors took a reverse genetic approach based on PCR screening of an Arabidopsis T-DNA tagged population (Wassilewskija ecotype, WS) available at the University of Wisconsin (Sussman et al., 2000). An Arabidopsis mutant line defective in GPAT1, gpat1-1, with a T-DNA insertion interrupting GPAT1 gene was identified. Sequencing of PCR products generated from GPAT1-specific and T-DNA-specific primers placed the T-DNA insert at the end of exon I of the GPAT1 gene (FIG. 5A). The T-DNA insertion predicted a removal of a segment in the GPAT1 transcript encoding amino acid 297-585 where the critical conserved domains of acyltransferases are located. Homozygous (gpat1-1/gpat1-1) and heterozygous (GPAT1/gpat1-1) lines were distinguished through PCR analysis using GPAT1 and T-DNA specific primers to generate fragments diagnostic of wild type (Wt) and gpat1-1 alleles. Analyses of southern blots probed with the BAR gene present in the T-DNA element resulted in the detection of a single hybridization band in gpat1-1 genomic DNA (FIG. 5B), suggesting that gpat1-1 harbors a single T-DNA insert. Northern blot analyses of RNA from the homozygous line revealed that GPAT1 mRNA was absent in gpat1-1. However, a short transcript, most likely representing a truncated GPAT1, was detected at a very low level (FIG. 5C). Taken together, the results suggest that gpat1-1 represents a null mutant with a single T-DNA insert disrupting GPAT1.

### Example 4

### Impact of GPAT Deficiency to Lipid Biosynthesis in Flower Tissues

Neither the homozygous nor the heterozygous line was found to be impaired in growth and development at vegetative stages. There were no changes in fatty acid content or composition in root or rosette leaf tissues. The inventors also analyzed glycerolipid profile in flower tissues where GPAT1 was expressed at a very high level. Total lipid extract from flower bud was separated into two fractions, one containing mainly TAGs and the other comprising of polar lipids (PL) that included phospholipid and glycoglycerolipid. The lipid content and fatty acid compositions of these two fractions are shown in Table I. There was a general reduction of lipid content in flower bud: TAG and PL were all reduced in gpat1-1 to approximately 90% of the Wt plants. In addition, fatty acid composition changes were also detected. TAG from gpat1-1had a dramatically reduced proportion of palmitic acid (16:0), whereas the proportions of polyunsaturated linoleic (18:2) and linolenic (18:3) acid increased. The miniscule amount of tissue available from Arabidopsis flower bud made it impractical to accurately analyze lipid content of various organs.

### Example 5

### Impact of GPAT Deficiency on Seed Yield and Storage Lipid Biosynthesis

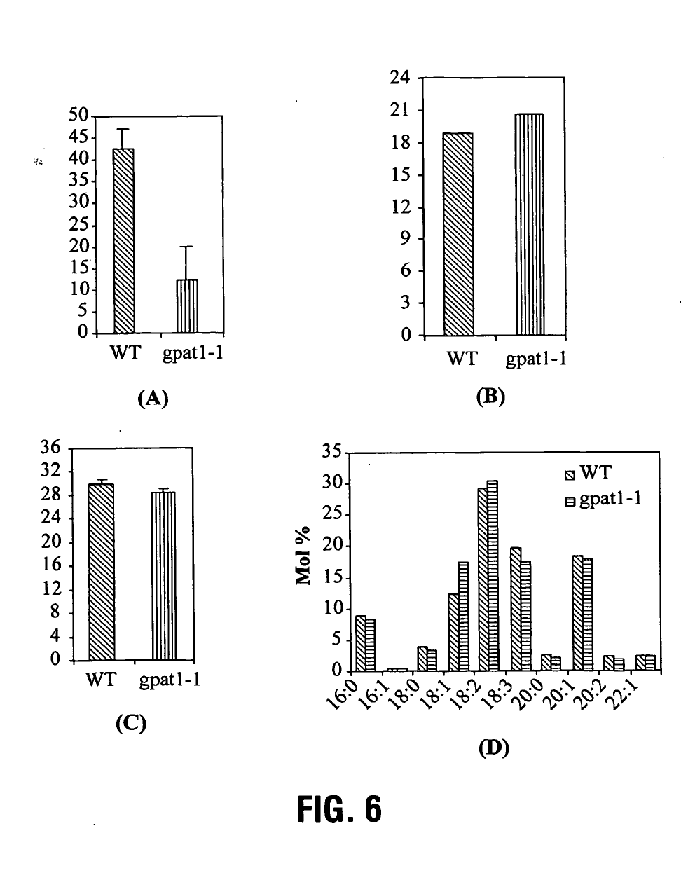

It was observed that, although plant growth and flowering were normal in gpat1-1, the seed yield was severely affected as indicated by the reduced size of most of its mature siliques. The heterozygous plants (GPAT1/gpat1-1) were fully fertile and produced normal siliques as in the Wt plants. The effect of gpat1-1 on seed yield in the homozygous lines (gpat1-1/gpat1-1) was studied by systematically examining the number of seeds produced in siliques from Wt and gpat1-1 plants grown in parallel under the same conditions. The average seed yield per silique in gpat1-1 was decreased by more than 72% (FIG. 6A). Also with regard to seed production was a significant difference in seed yield along the inflorescence axis. The siliques formed early on the inflorescence had one or no seed at all, whereas the siliques developed later contained up to 29 seeds. In comparison, the Wt produced 40 seeds per silique. Thus, there was a partial recovery of seed set as inflorescence development proceeded in the mutant.

Seeds of the gpat1-1 homozygous line allowed the inventors to examine the effect of GPAT 1 deficiency on storage lipid accumulation in mature seed. Repeated seed weight measurements revealed that gpat1-1 seeds were larger than those of Wt. Calculations from the total weight of up to 1000 seeds indicated that gpat1-1 had a seed weight of 20.55 μg/seed, while the Wt seed was 18.90 μg/seed. Thus, there was an increase of almost 9% in total mass in gpat1-1 seed (FIG. 6B). The significant increase in seed weight was contrasted by a decrease in the oil content of gpat1-1 , i.e. a 5.7% reduction as compared to Wt (FIG. 6C). Moreover, alterations of fatty acid composition in seed storage lipid also occurred. The most dramatic change involved oleic acid (18:1), which increased from 12.3% to 17.0%. A moderate but consistent increase in linoleic acid (18:2) was also observed, followed by a reduction in stearic acid (18:0), linolenic acid (18:3) and the very long chain eicosenoic acid (20: 1) to varying degrees (FIG. 6D).

### Example 6

### Identification of Other GPAT Homologs from Plants

To identify other GPAT isoforms from Arabidopsis and orthologs in other plant species, the inventors searched the database for related sequences. GPAT1 and GPAT2 are predicted to be members of an extensive group of membrane protein family from plants. Arabidopsis has at least 7 members (GPAT1; GPAT2; GPAT3 (At1g01610); At4g01950; At5g06090; At3g11430; At1g02390), and there are also members identified from rice (AP002869.1; AP000816.1; AP002883.2) and sorghum (AAL73535). As in GPAT1 and GPAT2, the deduced amino acid sequences of these GPAT-related proteins contain the acyltransferase domains: N/T-H-R-T-X-UM-D-P 40 (SEQ ID NO: 7), L/I-S/A-P-I/L-P/K-T/A-V/F-R/A/S-L-T/K-R-X-R (SEQ ID NO: 8), G-D-L-V-V/I-C/Y-P-E-G-T-T-C-R-E-P-F/Y-L-L-R-F-S (SEQ ID NO: 9), I-V-P-V-A (SEQ ID NO: 10). The functional domains are arranged in the same order and separated by similar distances, at the regions close by the C-termini. Moreover, there are at least four more conserved sequence domains easily identifiable. These include the F-X-Y-F/Y-M/F-L/V-V-A-F/L/I-E (SEQ ID NO: 11) motif, the I-F-H-D/E-G-R-L-V/A (SEQ ID NO: 12) motif, the D-P-X-F-F/A-F/L-M/L-N/D-P-X-P (SEQ ID NO: 13) motif and the F-E-C-T-X-F/L-T-R-K/R-D-K-Y (SEQ ID NO: 14) motif. Thus, the conserved sequence regions extend well beyond the previously defined function domains. Of additional significance, using trans-membrane domain prediction program TopPred2 and TMpred, all members were predicted to possess two membrane-spanning regions corresponding to aa129-aa151 and aa341-aa363 in GPAT1.

Materials and Methods

Yeast Strains and Culture Conditions

The haploid gene disruption strain gat1Δ(BY4742, Matα, his3`1, leu2`0, lys2`0, ura3`0, YKR067w::kanMX4) was purchased from Euroscarf. Cells were cultured at 30° C. in YPD medium containing 1% Bacto-yeast extract, 2% Bacto-peptone, and 2% glucose (Sigma).

Identification of GPAT1 and GPAT2 Gene from Arabidopsis

Database searches using BLAST program (Altschul et al., 1990) with sequences derived from conserved regions of the yeast GPATs and other membrane-bound acyltransferases identified a hypothetical gene encoding a polypetide of 585 aa in Arabidopsis. It contained the segments similar to the previously recognized acyltransferase motifs. This sequence, designated GPAT1, was amplified using the single strand cDNA transcribed from total RNA of Arabidopsis siliques and cell suspension. The primers used for polymerase chain reactions were derived from Arabidopsis genome sequences. The generated double strand cDNA was directly cloned into vector pYES2.1/V5-His-TOPO (Invitrogen), and the resulting plasmid GPAT1-pYES2.1/V5-His-TOPO was transformed into TOP-10 cells (Invitrogen). The GPAT1 cDNA was sequenced using an automated DNA sequencer (Applied Biosystems 373), and compared with the corresponding genomic sequence.

Computer Analysis of the GPAT1 and GPAT2 Sequence

The amino acid sequence deduced from the GPAT1 cDNA was analyzed for hydropathy profiles using the Kyte-Doolittle algorithm (DNAstar). The transmembrane domains of the sequence were predicted by TopPred2 program (von Heijne, 1992) and TMpred program (Hoffmann and Stoffel, 1993).

Heterologous Expression of the GPAT1 Gene in Yeast

Plasmid GPAT1-pYES2.1/V5-His-TOPO or the control vector was introduced into yeast Δgat1 strain (Zheng and Zou, 2001). To over-express the GPAT1 gene in yeast, a single colony containing GPAT1-pYES2.1/V5-His-TOPO was inoculated in 10 ml SD-uracil medium with 2% glucose and 0.25% glycerol. After incubation at 28° C. for 24 hours, the cells were harvested by centrifugation at 1500×g for 5 min, and resuspended in SD-uracil medium with1% raffinose, 2% galactose and 0.25% glycerol (SD induction medium). The cells were then diluted with 50 ml of SD induction medium to obtain a cell density of OD₆₀₀=0.5. After incubation at 16° C. for 40 hours, the cells were collected and yeast homogenates were prepared as described previously (Zheng and Zou, 2001), except that the extraction buffer contained 50 mM Hepes, pH 7.0, 2 mM EDTA, 1 mM DTT, 10% glycerol.

Enzyme Assays

GPAT activity was assayed at 30° C. for 10 minutes in a 200 μl reaction mixture containing 40 mM Hepes (pH 7.0), 400 μM [¹⁴C] glycerol-3-phosphate (2.5 nCi/nmol), 67.5 μM stearyol-CoA or other fatty acyl donors, 1 mM DTT, 5 mM EDTA, and 2.5 mg. mL⁻¹ BSA. The reaction mixture was stopped and products were extracted as previously described (Zheng and Zou, 2001). The formed products were subjected to scintillation counting for radioactivity and TLC analysis as described (Zheng and Zou, 2001).

Lipid Analysis

Total lipids from the tissues of Wt and mutant plants were extracted using the procedure of Bligh and Dyer (1959). Separation of neutral lipids and polar lipids was performed using Sep-Pak silica column (Waters) according to the procedure of Uemura et al. (1995). Individual neutral lipids, including triacylglycerol, were resolved on TLC plates (‘Baker’ Si 250-PA, UK) in a developing solvent of hexane/diethyl ether/acetic acid (80:20:2, v/v), and identified by co-migration with known standards. Seed oil was isolated as described (Zou et al., 1999). The isolated lipids were transmethylated with methanolic-HCl and quantified by gas chromatography as described previously (Zheng and Zou, 2001).

RNA Analyses

RNA extraction and northern analysis were conducted as described (Zheng et al., 2001). The cDNA fragment of GPAT1 gene was used as the probe for hybridization.

Screening of the gpat1 Mutant Allele

A total of 72,960 T-DNA tagged Arabidopsis (ecotype WS) plants generated at the Arabidopsis Knockout Facility of the University of Wisconsin were screened by PCR. The T-DNA left border primer (5′-CATTTTATAATAACGCTGCGGACATCTAC-3′-SEQ ID NO: 15) was selected for screening in combination with each of two primers, (5′-CTTCTCTCTCTACGCCATAGCTAT GGTTT-3′-SEQ ID NO: 16) and (5′-GCACTAAAGCGACAGGTT GAGATTATGGA-3′-SEQ ID NO: 17), designed according to sequences corresponding to the 5′ and 3′ end of the gene. The PCR products generated were subjected to southern analysis, and those which tested positive and were of appropriate size, were cloned into pCR2.1TOPO vector (Invitrogen) and sequenced to confirm insertion of T-DNA into the GPAT1 gene. To determine the T-DNA insertion number, southern analysis was conducted using the BAR gene of the T-DNA cassette as a probe to the genomic DNA isolated from the T2 homozygotes.

Sequence Listing Free Text

SEQ ID NO: 1 GPAT1 gene sequence
SEQ ID NO: 2 GPAT2 gene sequence
SEQ ID NO: 3 GPAT3 gene sequence
SEQ ID NO: 4 GPAT1 deduced protein sequence
SEQ ID NO: 5 GPAT2 deduced protein sequence
SEQ ID NO: 6 GPAT3 deduced protein sequence
SEQ ID NO: 7 Motif 1
SEQ ID NO: 8 Motif 2
SEQ ID NO: 9 Motif 3
SEQ ID NO: 10 Motif 4
SEQ ID NO: 11 Motif 5
SEQ ID NO: 12 Motif 6
SEQ ID NO: 13 Motif 7
SEQ ID NO: 14 Motif 8
SEQ ID NO: 15 Primer 1
SEQ ID NO: 16 Primer 2

SEQ ID NO: 17 Primer 3

TABLE I
Fatty acid compositions of the lipids from flower buds of Arabidopsis gpat1
mutants and wild-type (WT) plants.

| Lipid | | | Proportion of fatty acid | | | | | | | | |
|---|---|---|---|---|---|---|---|---|---|---|---|
| fraction | | Total lipid | 14:0 | 16:0 | 16:1 | 16:3 | 18:0 | 18:1 | 18:2 | 18:3 | 20:0 |
| μmol fatty acid g⁻¹ fresh wt | | | mol % | | | | | | | | |
| TAG | WT | 0.86 ± 0.01 | 1.28 | 23.86 | 0.58 | 1.02 | 2.05 | 4.36 | 23.40 | 42.51 | 0.55 |
| | gpat1 | 0.77 ± 0.08 | 1.29 | 18.44 | 0.66 | 0.96 | 1.74 | 5.04 | 26.80 | 44.40 | 0.46 |
| PL | WT | 3.40 ± 0.60 | nd | 22.06 | nd | 1.30 | 2.94 | 4.83 | 25.84 | 42.65 | nd |
| | gpat1 | 3.05 ± 0.20 | nd | 21.97 | nd | 0.51 | 2.27 | 5.02 | 27.27 | 42.27 | nd |

PL, polar lipids;
TAG, triacylglycerol
nd, not detectable.

### REFERENCES

Allen, D. and Kallen, K. J. (1993) Transport of lipids to the plasma membrane in animal cells. Prog. Lipid Res., 12, 195-219.
Altschul, S. F., Gish, W., Miller, W., Myers, E. W. and Lipman, D. J. (1990) Basic local alignment search tool. J. Mol. Biol., 215, 403-410.
Athenstaedt, K. and Daum, G. (1999) Phosphatidic acid, a key intermediate in lipid metabolism. Eur. J Biochem., 266, 1-16.
Barfor, M., Stobart, A. K. and Stymne, S. (1990) Properties of the glycerol acylating enzyme in microsomal preparations from the developing seeds of safflower (Carthamus tinctorius) and turnip rape (Brassica campestris) and their ability to assemble cocoa-butter type fats. J. Amer. Oil Chem. Soc., 67, 217-225.
Bertrams, M. and Heinz, E. (1976) Experiments on enzymatic acylation of sn-glycerol 3-phosphate with enzyme preparations from pea and spinach leaves. Planta, 132, 161-168.
Bertrams, M. and Heinz, E. (1981) Positional specificity and fatty acid selectivity of purified sn-glycerol 3-phosphate acyltransferases from chloroplasts. Plant Physiol., 68, 653-657.
Bligh, E. G. and Dyer, W. J. (1959) A rapid method of total lipid extraction and purification. Can. J Biochem. Physiol, 37, 911-917.
Browse, J., Warwick, N., Somerville, C. R. and Slack, C. R. (1986) Fluxes through the prokaryotic and eukaryotic pathways of lipid synthesis in the 16:3 plant Arabidopsis thaliana. Biochem. J., 235, 25-31.
Dircks, L. and Sul, H. S. (1999) Acyltransferases of de novo glycerophospholipid biosynthesis. Prog Lipid Res., 38, 461-79.

Dörmann, P. and Benning, C. (2002) Galactolipids rule in seed plants. Trends Plant Sci., 7, 112-118.
Eccleston, V. and Harwood, J. L (1995) Solubilization, partial purification and properties of acyl-CoA: glycerol-3-phosphate acyltransferase from avocado (Persea americana) fruit mesocarp. Biochem. Biophy. Acta, 1257, 1-10.
Frentzen, M (1990) Comparison of certain properties of membrane bound and solublized acyltransferase activities of plant microsomes. Plant Sci., 69, 39-48.
Fritz, P. J., Kauffman, J. M., Robertson, C. A. and Wilson, M. R. (1986) Cocoa butter biosynthesis, purification and characterization of a soluble sn-glycerol-3-phosphate acyltransferase from cocoa seeds. J. Biol. Chem., 261, 194-199.
Griffiths, G. Stobart, A. K. and Stymne, S. (1985) The acylation of sn-glycerol-3-phosphate and the metabolism of phosphatidate in microsomal preparations from the developing cotyledons of safflower (Cathamus tinctorius L.) seed. Biochem. J., 230, 379-388.
Hares, W. and Frentzen, M. (1991) Substrate specificities of the membrane-bound and partially purified microsomal acyl-CoA: 1-acylglycerol-3-phosphate acyltransferase from etiolated shoots of Pisum sativum (L.). Planta, 185, 124-131.
Harwood, J. L. and Page, R. A (1994) Biochemistry of oil synthesis. In Murphy, D. J. (ed) Designer oil crops. VCH, Weinheim, pp 165-194.
Heinz, E. and Roughan, P. G. (1983) Similarities and differences in lipid metabolism of chloroplasts isolated from 18:3 and 16:3 plants. Plant Physiol., 72, 273-279.
Hofmann, K. and Stoffel, W. (1993) TMbase—A database of membrane spanning proteins segments. Biol. Chem. Hoppe-Seyler, 374, 166.
Ichihara, K. (1984) sn-glycerol-3-phosphate acyltransferase in a particulate fraction from maturing safflower seeds. Arch. Biochem. Biophy., 232, 685-698.
Ichihara, K., Asashi, T. and Fujii, S. (1987) 1-acylglycerol-sn-glycerol-3-phosphate acyltransferase in maturing safflower seeds and its contribution to the non-random fatty acid distribution of triacylglycerol. Eur. J Biochem. 167, 339-347.
Ishizaki, O., Nishida, I., Agata, K., Eguchi, G. and Murata, N. (1988) Cloning and nucleotide sequence of cDNA for the plastid glycerol-3-phosphate acyltransferase from squash. FEBS Lett., 238, 424-30.
Katavic, V., Reed, D. W., Taylor, D. C, Giblin, E. M., Barton, D. L., Zou, J., MacKenzie, S. L., Covello, P. S. and Kunst, L. (1995) Alteration of seed fatty acid composition by an ethyl methanesulfonate-induced mutation in Arabidopsis thaliana affecting diacylglycerol acyltransferas activity. Plant Physiol., 108, 399-409.
Kunst, L., Browse, J. and Somerville, C. (1988) Altered regulation of lipid biosynthesis in a mutant of Arabidopsis deficient in chloroplast glycerol-3-phosphate acyltransferase activity. Proc. Natl. Acad. Sci. USA, 85, 4143-4147.
Lewin, T. M., Wang, P. and Coleman, R. A. (1999) Analysis of amino acid motifs diagnostic for the sn-glycerol-3-phosphate acyltransferase reaction. Biochemistry, 38, 5764-5771.
Manaf, A. M. and Harwood, J. L. (2000) Purification and characterization of acyl-CoA: glycerol 3-phosphate acyltransferase from oil palm (Elaeis guineensis) tissues. Planta, 210, 318-328.
Moore, Jr. T. S. (1982) Phospholipid biosynthesis. Ann. Rev. Plant Physiol., 33, 235-259.
Nishida, I., Tasaka, Y., Shiraishi, H. and Murata, N. (1993) The gene and the RNA for the precursor to the plastid-located glycerol-3-phosphate acyltransferase of Arabidopsis thaliana. Plant Mol Biol, 21, 267-77.
Roughan, P. G. and Slack, C. R. (1982) Cellular organization of glycerolipid metabolism. Annu. Rev. Plant Physiol., 33, 97-132.
Roughan, P. G. and Slack, C. R. (1984) Glycerolipid synthesis in leaves. Trends Biochem. Sci. 9, 383-386.
Routaboul, J. M., Benning, C., Bechtold, N., Caboche, M. and Lepiniec, L (1999) The TAG1 locus of Arabidopsis encodes for a diacylglycerol acyltransferase. Plant Physiol. Biochem., 37, 831-840.
Schrauwen, J. A. M., Mettenmeyer, T., Croes, A. F. and Wullems, G. J. (1996) Tapetum-specific genes: what role do they play in male gametophyte development? Acta Bot. Neerl., 45, 1-15.
Sun, C., Cao, Y. Z. and Huang, A. H. C (1988) Acyl coenzyme A preference of the glycerol phosphate pathway in the microsomes from the maturing seeds of palm, maize and rapeseed. Plant Physiol., 88, 56-60.
Sussman, M. R., Amasino, R. M., Young, J. C., Krysan, P. J. and Austin-Phillips, S. (2000) The Arabidopsis Knockout Facility at the University of Wisconsin-Madison. Plant Physiol., 124, 1465-1467.
von Heijne, G. (1992) Membrane Protein Structure Prediction, Hydrophobicity Analysis and the Positive-inside Rule. J. Mol. Biol., 225,487-494.
Wilkison, W. O. and Bell, R. M. (1997) sn-glycerol-3-phosphate acyltransferase from Escherichia coli. Biochem. Biophy. Acta., 1348, 3-9.
Zheng, Z., Uchacz, T. M., and Taylor, J. L. (2001) Isolation and characterization of novel defence-related genes induced by copper, salicylic acid, methyl jasmonate, abscisic acid and pathogen infection in Brassica carinata. Mol. Plant Pathol, 2, 159-169.
Zheng, Z. and Zou, J. (2001) The initial step of the glycerolipid pathway, identification of glycerol 3-phosphate/dihydroxyacetone phosphate dual substrate acyltransferases in Saccharomyces cerevisiae. J. Biol. Chem., 276, 41710-41716.
Zou, J., Wei, Y., Jako, C., Kumar, A., Selvaraj, G. and Taylor, D. C. (1999) The Arabidopsis thaliana TAG1 mutant has a mutation in a diacylglycerol acyltransferase gene. Plant J., 19, 645-653.

1. An isolated nucleotide sequence, characterized in that said sequence encodes a cytoslic ER-based glycerol-3-phosphate acyltransferase protein, or a fragment thereof.

2. An isolated nucleotide sequence according to claim 1, characterized in that said isolated nucleotide sequence is selected from:
a) a cytoslic ER-based glycerol-3-phosphate acyltransferase gene as shown in SEQ ID NO: 1, 2, or 3, or a complement thereof;
b) a nucleotide sequence encoding a peptide with at least 40% identity to a peptide encoded by the nucleotide sequence of b);
wherein said nucleotide sequence or complement thereof encodes a protein or a part thereof, that alters a lipid metabolism of a transgenic plant exogenously expressing said nucleotide sequence compared to an unmodified plant.

3. An isolated nucleotide sequence according to claim 2 chracterized in that said nucleotide sequence has at least 70% identity to the cytoslic ER-based glycerol-3-phosphate acyltransferase gene shown in SEQ ID NO: 1, 2 or 3 or a complement thereof.

4. An isolated nucleotide sequence according to claim 2 characterized in that said nucleotide sequence has at least 90% identity to the cytoslic ER-based glycerol-3-phosphate acyltransferase gene shown in SEQ ID NO: 1, 2 or 3 or a complement thereof.

5. An isolated nucleotide sequence according to claim 2 characterized in that said nucleotide sequence has at least 95% identity to the cytoslic ER-based glycerol-3-phosphate acyltransferase gene shown in SEQ ID NO: 1, 2 or 3, or a complement thereof.

6. An isolated nucleotide sequence according to claim 1, characterized in that said isolated nucleotide sequence is selected from the group consisting of:
a) a cytoslic ER-based glycerol-3-phosphate acyltransferase gene according to SEQ ID NO: 1, 2 or 3, or a complement thereof;
b) a nucleotide sequence that hybridizes under stringent conditions to the nucleotide sequence of a);
wherein said nucleotide sequence or complement thereof encodes a protein or part thereof that alters lipid metabolism of a transgenic plant exogenously expressing said nucleotide sequence compared to an unmodified plant.

7. An isolated nucleotide sequence of claim 2, characterized in that expression of said nucleotide sequence confers on said transgenic plant an increased level of TAG compared to an unmodified plant.

8. An isolated nucleotide sequence of claim 2, characterized in that expression of said nucleotide sequence confers on said transgenic plant an altered growth potential selected from the group consisting of: faster growth rate, slower growth rate, larger biomass, and smaller biomass,

9. An isolated nucleotide sequence according to claim 2 characterized in that the nucleotide sequence is derived from an Arabidopsis plant.

10. An isolated nucleotide sequence according to claim 2 characterized in that expression of said nucleotide sequence in a plant causes said plant to have seeds with an increased TAG level compared to an unmodified plant.

11. An isolated and purified peptide characterized in that said isolated and purified peptide is encoded by the nucleotide sequence according to claim 1.

12. A DNA expression cassette characterized in that said DNA expression cassette comprises the nucleotide sequence according to claim 2, operably linked to a promoter.

13. A construct characterized in that said construct comprises a vector and the nucleotide sequence according to claim 2.

14. A construct according to claim 13 characterized in that said nucleotide sequence is operably linked to a promoter.

15. A construct of according to claim 14 characterized in that said promoter is selected from the group consisting of: a constitutive promoter, an inducible promoter, an organ specific promoter, a strong promoter, a weak promoter, and a stress induced promoter.

16. A plant cell characterized in that said plant cell is transformed with the construct according to claim 13.

17. A transgenic plant characterized in that said transgenic plant is derived from regeneration of said plant cell according to claim 16.

18. A transgenic plant according to claim 17 characterized in that said transgenic plant is selected from a species of grain producing crop, a fruit or vegetable species, and a horticultural species.

19. A transgenic plant according to claim 18 characterized in that said transgenic plant is a species selected from the group consisting of: canola, safflower, sunflower, and olive.

20. A method of genetically modifying a plant, characterized in that the method comprising the steps of:
(a) introducing into a plant cell capable of being transformed and regenerated into a whole plant a construct comprising, in addition to the DNA sequences required for transformation and selection in plants, a nucleotide sequence according to claim 2, operably linked to a promoter; and
(b) recovery of a plant which contains said nucleotide sequence.

21. A method according to claim 20 characterized in that said plant exhibits an altered lipid metabolism compared to an unmodified plant.

22. A method according to claim 20 characterized in that said plant exhibits an increase in levels of TAG compared to an unmodified plant.

23. A method according to claim 20 characterized in that said plant exhibits an altered growth potential selected from the group consisting of: faster growth rate, slower growth rate, larger biomass, and smaller biomass,

24. A method according to claim 20 characterized in that said nucleotide sequence is oriented in a sense direction relative to a promoter.

25. A method according to claim 20 characterized in that said nucleotide sequence is oriented in an antisense direction relative to a promoter.

26. A method of identifying and isolating a DNA sequence substantially homologous to the nucleotide sequence of claim 1, characterized in that said method comprising the steps of:
synthesizing a degenerate oligonucleotide primer than can hybridize to the nucleotide sequence of claim 1 under stringent conditions;
labelling said degenerate oligonucleotide primer; and
using said labelled degenerate oligonucleotide primer as a probe to screen a DNA library for said substantially homologous DNA sequence.

27. A DNA sequence characterized in that said DNA sequence is obtainable by the method according to claim 26.

28. A pair of primers characterized in that said primers hybridize to selected portions of the nucleotide sequence of claim 1, for amplifying a region of DNA between said primers by polymerase chain reaction.

29. Use of an isolated nucleotide sequence according to claim 1, characterized in that said use is for generating a transgenic plant that exhibits an altered lipid metabolism compared to an unmodified plant.

30. Use of an isolated nucleotide sequence according to claim 1, characterized in that said use is for generating a trangenic plant having that exhibits an altered growth potential compared to an unmodified plant.

31. A method of producing a transgenic plant with altered lipid metabolism and/or altered lipid levels compared to an unmodified plant, characterized in that the method comprising the steps of:
(a) introducing into a plant cell capable of being transformed and regenerated into a whole plant a construct comprising, in addition to the DNA sequences required for transformation and selection in plants, an isolated nucleotide sequence according to claim 1 operably linked to a promoter; and
(b) recovery of a plant which contains said nucleotide sequence and has a modified lipid metabolism and/or growth potential compared to an unmodified plant.

32. A method according to claim 31, characterized in that said nucleotide sequence encodes a peptide having at least 40% identity to the peptide encoded by SEQ ID NO: 1, 2 or 3, or a part thereof, or a complement thereof.

33. A method according to claim 31, characterized in that said nucleotide sequence encodes a peptide having at least 70% identity to the peptide encoded by SEQ ID NO: 1, 2 or 3, or a part thereof, or a complement thereof.

34. A method according to claim 31, characterized in that said nucleotide sequence encodes a peptide having at least 90% identity to the peptide encoded by SEQ ID NO: 1, 2 or 3, or a part thereof, or a complement thereof.

35. A method according to claim 31, characterized in that said nucleotide sequence encodes a peptide having at least 95% identity to the peptide encoded by SEQ ID NO: 1, 2 or 3, or a part thereof, or a complement thereof

36. A method according to claim 31, characterized in that said nucleotide sequence encodes a peptide having at least 99% identity to the peptide encoded by SEQ ID NO: 1, 2 or 3, or a part thereof, or a complement thereof.

37. A method according to claim 31, characterized in that said nucleotide sequence is the nucleotide sequence indicated in SEQ ID NO: 1, 2 or 3, or a part thereof, or a complement thereof, or a nucleotide sequence that binds under stringent conditions to the nucleotide sequence indicated in SEQ ID NO: 1, 2 or 3, or a part thereof, or a complement thereof

38. A method according to claim 31, characterized in that said nucleotide sequence is expressed in a sense direction for complementary inhibition of an endogenous cytoslic ER-based glycerol-3-phosphate acyltransferase gene in said plant, said plant having a decreased lipid levels and/or a decreased growth potential compared to an unmodified plant.

39. A method according to claim 38, characterized in that said nucleotide sequence is a mutated cytoslic ER-based glycerol-3-phosphate acyltransferase gene.

40. A method according to claim 31, characterized in that said nucleotide sequence is expressed in an antisense direction for antisense inhibition of an endogenous cytoslic ER-based glycerol-3-phosphate acyltransferase gene of said plant, said plant having decreased lipid levels and/or a decreased growth potential compared to an unmodified plant.

41. A method according to claim 31, characterized in that said nucleotide sequence is overexpressed in a sense direction, said plant having an increased level of lipids and/or an increased growth potential compared to an unmodified plant.

42. A method according to claim 31, characterized in that said promoter comprises a transcriptional regulatory region normally in operable association with an endogenous cytoslic ER-based glycerol-3-phosphate acyltransferase gene or homologue thereof.

43. A method according to claim 31, characterized in that said promoter comprises a transcriptional regulatory region that is not normally in operable association with an endogenous cytoslic ER-based glycerol-3-phosphate acyltransferase gene or homologue thereof.

44. A method according to claim 31, characterized in that said promoter is selected from the group consisting of: a constitutive promoter, an inducible promoter, an organ specific promoter, a strong promoter, a weak promoter, and an endogenous cytoslic ER-based glycerol-3-phosphate acyltransferase promoter.

45. A method of identifying a plant that has been successfully transformed with a construct, characterized in that the method comprises the steps of:
(a) introducing into plant cells capable of being transformed and regenerated into whole plants a construct comprising, in addition to the DNA sequences required for transformation and selection in plants, a nucleotide sequence according to claim 1 and encoding at least part of a cytoslic ER-based glycerol-3-phosphate acyltransferase gene product, operably linked to a promoter;
(b) regenerating said plant cells into whole plants; and
(c) inspecting the plants to determine those plants successfully transformed with said construct, and expressing said nucleotide sequence, said plants having an altered lipid content and/or an altered growth potential compared to an unmodified plant.

46. A method according to claim 45, characterized in that said construct is bicistronic and further comprises a second DNA expression cassette for generating a transcript unrelated to said nucleotide sequence derived from a cytoslic ER-based glycerol-3-phosphate acyltransferase gene.

47. A transgenic plant characterized in that said transgenic plant is generated by the method according to claim 31.

48. A bicistronic vector characterized in that said bicistronic vector comprises a first nucleotide sequence according to claim 2 operatively linked to a first tissue-specific promoter, and a second nucleotide sequence according to claim 2 operatively linked to a second tissue-specific promoter.

49. The bicistronic vector according to claim 48, characterized in that expression of said vector in a transgenic plant induces alternative lipid metabolism and growth potential characteristics in different tissues of said plant according to said first and second nucleotide sequences and said operatively linked first and second promoters.

50. A bicistronic vector according to claim 49, characterized in that said first nucleotide sequence is oriented in a sense direction relative to said first promoter, and said second nucleotide sequence is oriented in an antisense direction relative to said second promoter.

51. A bicistronic vector according to claim 49, characterized in that said first nucleotide sequence encodes a biologically active form of a cytoslic ER-based glycerol-3-phosphate acyltransferase protein or a part thereof, and said second nucleotide sequence encodes a biologically inactive form of a cytoslic ER-based glycerol-3-phosphate acyltransferase protein or a part thereof.

52. A transgenic plant characterized in that said transgenic plant is transformed with a bicistronic vector according to claim 48.

53. A cytoslic ER-based glycerol-3-phosphate acyltransferase protein, or a fragment thereof encoded by the nucleotide sequence according to claim 1 that exhibits cytoslic ER-based glycerol-3-phosphate acyltransferase activity, characterized in that said protein comprises all of the peptide sequence motifs of: SEQ ID NO: 7, 8, 9, 10, 11, 12, 13, or 14.

54. A nucleotide sequence encoding a cytosolic ER-based glycerol-3-phosphate acyltransferase protein or fragment thereof according to claim 53, or a complement thereof.

55. A DNA expression cassette characterized in that said DNA expression cassette includes the nucleotide sequence according to claim 54, operably linked to a promoter.

56. A construct, characterized in that said construct includes the nucleotide sequence according to claim 54, operably linked to a promoter.

57. A plant cell, characterized in that said plant cell includes a DNA expression cassette according to claim 55.

58. A plant seed, characterized in that said plant seed includes a DNA expression cassette according to claim 55.

59. A plant having a genome, characterized in that said genome includes an introduced nucleotide sequence according to claim 54.


SEQUENCE LISTING (17 sequences)

SEQ ID NO: 1 - DNA, 1769 bp, Arabidopsis sp.
  misc_feature: GPAT 1 gene sequence
gctatggttttaccagagcttctcgtgattctagcagaatgggttctttaccgtcttcttgctaattcatgttacagagctgctagaaagctcagaggctatggtttccagttaaaaaatctcccaagcctctccaaaacacaatctctacacaacaattctcagcatcatcttcataaccatcatcaacaaaatcaccctaaccaaacccttcaagattctctagaccctctgttcccaagccttacaaaataccaagaacttcttctagacaaaaacagagcatgctctgtttcgtctgatcattatcgtgatacctttttctgcgatatcgatggtgttttattaagacaacattcgagcaaacacttccacacgttcttcccttacttcatgctcgtggcttttgaaggaggatcgatcatcagagcgatccttctcttgctctcctgttctttcctttggactctgcaacaagaaaccaaactcagagttctctctttcataaccttctctggtcttagagtcaaagacatggacaacgtttctagatcggttctaccaaaattcttcctcgagaacttgaatatccaagtttacgacatctgggctcgaactgagtactccaaagttgtgttcacgagcttgccgcaagtgctggtggagaggttcctcagagaacatctcaacgctgatgatgtgattgggactaaactgcaagaaataaaagtcatgggaaggaaattctacacaggcctagcttctggttctggtttcgtcctgaaacataaatcagctgaagactactttcttgatagcaagaagaagcctgcacttggaattggaagctcttcaagcccacaagatcacatcttcatctccatttgcaaagaagcttatttttggaatgaagaagagtccatgagcaagaacaacgcattgcctagagaaagatacccaaagccactgatattccacgacgggagattggctttcttgccaacaccattagctacattagccatgttcatatggctccctatcggatttttactcgctgtcttcaggatctcggtaggagtctttcttccttaccatgtagccaatttcttggcttctatgtccggtgttaggatcactttcaagacccataatttgaacaacggtcgtccagagaaaggtaatagtggagtcttgtatgtatgcaaccataggaccctcctcgacccagttttcctcaccacttcgctgggcaagcccttgaccgcggttacttacagcctaagcaagttctccgagttcatagcacctctcaagacggttagccttaaaagagataggaagaaagacggtgaggcaatgcagagactattaagcaaaggagatctagtggtttgtccggagggaaccacatgtagagagccttatctactaaggttcagtcctttgttcgctgagttaactgaagacattgttcccgtggctgtagatgctcgagtgagcatgttctatgggacaactgcgagcgggctcaagtgtttagaccctatcttcttcctcatgaaccctaggccggtttattgtcttgagattttaaagaagttgccaaaagagatgacttgtgcaggaggcaagtcaagctttgaagttgcgaatttcatacagggagaacttgctagggttttgggatttgaatgcactaatctcacaaggagggataagtacttggttcttgctggaaatgaaggcattgtcagataaactattcc

SEQ ID NO: 2 - DNA, 1508 bp, Arabidopsis sp.
  misc_feature: GPAT2 gene sequence
taatgggagctcaggagaaacggcgccgtttcgagccgatatcaaagtgcgatgttaaggaccggtccaaccataccgtcgccgctgatctagacggaacactactaatctctcgtagcgccttcccttactatttcctcgtagccctcgaggcagggagcttgctccgagcgttgatcctacttgtgtccgtaccattcgtttatcttacgtacttgaccatctccgagactttagccatcaacgtctttgtcttcatcacgttcgcgggtctcaagatcggagacgttgagctagtggtccgttccgtcctcccgaggttctacgcggaggacgtgaggcccgatacctggcgtatcttcaacacgttcgggaaacggtacatagtaactgcgagtcctcgaattatggtcgagccgttcgtgaaaacattcctaggagttgataaagttcttggaacagagctaaaggtctccaaatcgggtcgggcaaccgggttcaccagaaacccaggtattctcgtcggtcagcacaaacgtgacgtcgttttgagagagtttggtagccttgcgtctgatttacctgatttggggctcggcgatagcatgacggaccacgacttcatgtccatctgcaaggaaggttacatggtgccacgtacgaaatgcgaaccattaccaagaaacaaactcttaagccccataatattccacgagggcagattagtccaacgcccaacgccgttagttgctctgttaactttcctctggcttcccgtcggtttcctcctctctatcatccgcgtctacacgaatattccgttaccggaacgtatcgcccgttacaactacaagcttactggcatcaagctagtcgtcaacggccaccctcctccgccgccaaaacctggccagccaggccatcttttggtctgcaaccaccgcaccgttctcgatcctgtggtcacagctgtcgcactcggccggaaaatcagctgcgtcacttacagcatcagcaagttctctgagctaatctcaccaatcaaagccgttgcgttgactcgtcaacgtgagaaagacgcagcgaacatcaagcgtcttttggaggaaggcgatctcgtgatatgtcccgagggaaccacgtgccgtgagcctttccttctccggtttagtgctcttttcgctgagctcacggaccggatcgttcccgtggcgatcaacacaaagcagagcatgttcaatggtaccaccacacgtggatacaagcttcttgatccttactttgcgttcatgaacccgaggccgacgtatgagatcacgttcctcaaacagattccagctgagctgacgtgtaaaggaggcaaatctccgatagaggttgcgaattacatacagagggttttgggaggaaccttaggttttgagtgcaccaatttcacaagaaaggataagtacgcaatgcttgctggtactgacggtagggttccggtgaagaaggagaagacgtga

SEQ ID NO: 3 - DNA, 1540 bp, Arabidopsis sp.
  misc_feature: GPAT3 gene sequence
gccgcaagaaagatgtctccggcgaagaagagccgaagttttcctccgataagcgagtgtaaaagcagagagtatgattcgatcgctgcggatctcgacgggactctgcttctgtcaagaagctctttcccttacttcatgctggtggccattgaagcaggtagtctcttccgtggattgatccttcttctttctcttcccatcgtcatcatcgcttatctcttcgtctccgaatctctcggaatccaaatccttatcttcatctccttcgccggtatcaagatcaagaacatcgaactcgtctctcgcgccgttcttacacggttttacgcggcggatgtgaggaaagatagttttgaggtgtttgataaatgtaagaagaggaaagtagtggtgactgcgaatccgatagtgatggttgagccattcgtcaaagactatttgggaggagataaagttttgggaacagagattgaagttaaccccaaaacgatgaaagctactggttttgtgaaaaagcctggtgttcttgttggtgatctcaagagattagccatcttgaaagagttcggcgatgactcaccggatcttggcctcggtgaccgcacctccgatcacgatttcatgtctatttgcaaggaaggttacatggtgcatgagaccaaatcagccacaacagtccctatagagagtctcaagaaccgcataatcttccatgatggccgtcttgtccaacgtccgaccccattaaacgccttaatcatttacctttggcttcctttcggcttcatgctctctgtcttccgcgtctacttcaacctccctttacccgaacgcttcgtccgctacacttacgagatcctcggcattcacctcacaatccgtggccaccgtcctccacctccttcccccggaaaacctggaaacctctacgttctcaaccaccgcaccgcccttgaccccattatcatcgccattgctctcggccgtaagatcacatgtgtcacttatagtgtctctcgcctctccctgatgctctcacccattcctgctgttgctctgacccgagaccgtgtcgctgacgcagcccgcatgaggcaactcctcgagaaaggtgatttggtgatctgtcctgaaggcacaacgtgtagagaaccatatctactaaggttcagtgctctatttgcagagctgagcgaccggatcgtgcctgtggccatgaactgcaagcaagggatgttcaacgggacgacagttagaggtgtgaaattctgggatccgtacttcttcttcatgaacccccgacctagctacgaggccactttcttggaccgtttgcccgaagaaatgacggtaaacggtggtggcaagactccttttgaggtggctaattacgttcagaaggtgatcggtggagttttggggttcgaatgtactgaacttacaaggaaggataagtatctgttgcttggaggcaacgacggaaaggttgagtctatcaataagaccaagtccatggagtaataatccagctagtctg

SEQ ID NO: 4 - protein, 585 aa, Arabidopsis sp.
  MISC_FEATURE: GPAT1 deduced protein sequence
MVLPELLVILAEWVLYRLLANSCYRAARKLRGYGFQLKNLPSLSKTQSLHNNSQHHLHNHHQQNHPNQTLQDSLDPLFPSLTKYQELLLDKNRACSVSSDHYRDTFFCDIDGVLLRQHSSKHFHTFFPYFMLVAFEGGSIIRAILLLLSCSFLWTLQQETKLRVLSFITFSGLRVKDMDNVSRSVLPKFFLENLNIQVYDIWARTEYSKVVFTSLPQVLVERFLREHLNADDVIGTKLQEIKVMGRKFYTGLASGSGFVLKHKSAEDYFLDSKKKPALGIGSSSSPQDHIFISICKEAYFWNEEESMSKNNALPRERYPKPLIFHDGRLAFLPTPLATLAMFIWLPIGFLLAVFRISVGVFLPYHVANFLASMSGVRITFKTHNLNNGRPEKGNSGVLYVCNHRTLLDPVFLTTSLGKPLTAVTYSLSKFSEFIAPLKTVSLKRDRKKDGEAMQRLLSKGDLVVCPEGTTCREPYLLRFSPLFAELTEDIVPVAVDARVSMFYGTTASGLKCLDPIFFLMNPRPVYCLEILKKLPKEMTCAGGKSSFEVANFIQGELARVLGFECTNLTRRDKYLVLAGNEGIVR

SEQ ID NO: 5 - protein, 501 aa, Arabidopsis sp.
  MISC_FEATURE: GPAT2 deduced protein sequence
MGAQEKRRRFEPISKCDVKDRSNHTVAADLDGTLLISRSAFPYYFLVALEAGSLLRALILLVSVPFVYLTYLTISETLAINVFVFITFAGLKIGDVELVVRSVLPRFYAEDVRPDTWRIFNTFGKRYIVTASPRIMVEPFVKTFLGVDKVLGTELKVSKSGRATGFTRNPGILVGQHKRDVVLREFGSLASDLPDLGLGDSMTDHDFMSICKEGYMVPRTKCEPLPRNKLLSPIIFHEGRLVQRPTPLVALLTFLWLPVGFLLSIIRVYTNIPLPERIARYNYKLTGIKLVVNGHPPPPPKPGQPGHLLVCNHRTVLDPVVTAVALGRKISCVTYSISKFSELISPIKAVALTRQREKDAANIKRLLEEGDLVICPEGTTCREPFLLRFSALFAELTDRIVPVAINTKQSMFNGTTTRGYKLLDPYFAFMNPRPTYEITFLKQIPAELTCKGGKSPIEVANYIQRVLGGTLGFECTNFTRKDKYAMLAGTDGRVPVKKEKT

SEQ ID NO: 6 - protein, 503 aa, Arabidopsis sp.
  MISC_FEATURE: GPAT3 deduced protein sequence
MSPAKKSRSFPPISECKSREYDSIAADLDGTLLLSRSSFPYFMLVAIEAGSLFRGLILLLSLPIVIIAYLFVSESLGIQILIFISFAGIKIKNIELVSRAVLTRFYAADVRKDSFEVFDKCKKRKVVVTANPIVMVEPFVKDYLGGDKVLGTEIEVNPKTMKATGFVKKPGVLVGDLKRLAILKEFGDDSPDLGLGDRTSDHDFMSICKEGYMVHETKSATTVPIESLKNRIIFHDGRLVQRPTPLNALIIYLWLPFGFMLSVFRVYFNLPLPERFVRYTYEILGIHLTIRGHRPPPPSPGKPGNLYVLNHRTALDPIIIAIALGRKITCVTYSVSRLSLMLSPIPAVALTRDRVADAARMRQLLEKGDLVICPEGTTCREPYLLRFSALFAELSDRIVPVAMNCKQGMFNGTTVRGVKFWDPYFFFMNPRPSYEATFLDRLPEEMTVNGGGKTPFEVANYVQKVIGGVLGFECTELTRKDKYLLLGGNDGKVESINKTKSME

SEQ ID NO: 7 - protein, 8 aa, Artificial sequence
  Motif 1
XHRTXXDP

SEQ ID NO: 8 - protein, 13 aa, Artificial sequence
  Motif 2
XXPXXXXXLXRXR

SEQ ID NO: 9 - protein, 21 aa, Artificial sequence
  Motif 3
GDLVXXPEGTTCREPXLLRFS

SEQ ID NO: 10 - protein, 5 aa, Artificial sequence
  Motif 4
IVPVA

SEQ ID NO: 11 - protein, 10 aa, Artificial sequence
  Motif 5
FXYXXXVAXE

SEQ ID NO: 12 - protein, 8 aa, Artificial sequence
  Motif 6
IFHXGRLX

SEQ ID NO: 13 - protein, 11 aa, Artificial sequence
  Motif 7
DPXFXXXXPXP

SEQ ID NO: 14 - protein, 12 aa, Artificial sequence
  Motif 8
FECTXXTRXDKY

SEQ ID NO: 15 - DNA, 29 bp, Artificial sequence
  Primer 1
cattttataataacgctgcggacatctac

SEQ ID NO: 16 - DNA, 29 bp, Artificial sequence
  Primer 2
cttctctctctacgccatagctatggttt

SEQ ID NO: 17 - DNA, 29 bp, Artificial sequence
  Primer 3
gcactaaagcgacaggttgagattatgga